(19) United States
(12) Patent Application Publication
(10) Pub. No.: US 2024/0279642 A1
(43) Pub. Date: Aug. 22, 2024

(54) METHODS, SYSTEMS, AND COMPOSITIONS OF GENERATING AND ANALYZING POLYPEPTIDE LIBRARIES

(71) Applicant: Protillion Biosciences, Inc., Burlingame, CA (US)

(72) Inventors: Curtis James LAYTON, San Bruno, CA (US); Pavanapuresan Pushpagiri VAIDYANATHAN, Redwood City, CA (US); Michael Roy GOTRIK, Redwood City, CA (US)

(21) Appl. No.: 18/570,580

(22) PCT Filed: Jun. 14, 2022
(86) PCT No.: PCT/US2022/033437

§ 371 Date: Dec. 14, 2023

Related U.S. Application Data
(60) Provisional application No. 63/210,905, filed on Jun. 15, 2021.

(51) Int. Cl. C12N 15/10; C40B 30/04; C40B 40/08; G16B 35/20

(52) U.S. Cl. CPC C12N 15/1062; C40B 30/04; C40B 40/08; G16B 35/20

(57) ABSTRACT

Methods, systems, and composition for the analysis of polypeptides and the generation of polypeptide libraries are disclosed. Analysis of polypeptide library may be used to generate polypeptide with particular characteristics. Antibodies with high affinities may be generated using the methods, systems and compositions disclosed.

### CROSS REFERENCE

This application claims priority to U.S. Provisional Application No. 63/210,905, filed Jun. 15, 2021, which is incorporated by reference herein in its entirety.

### BACKGROUND

Polypeptides may be used for various purposes such as therapeutics. Directed evolution or selection strategies may be used to identify polypeptides of interest. Methods of protein display may be used in conjunction with directed evolution. Directed evolution technique may use protein display to screen for polypeptides of interest. Directed evolution and screening technique may be effective at identifying polypeptide of interest but may inadvertently lose potentially valuable polypeptides due to the complexity of sequence space and the lack of sequence diversity.

### SUMMARY

Provided herein are methods, systems and compositions for analysis of large numbers of polypeptides. The methods, systems and compositions may allow for the generation of polypeptides with particular characteristics. The methods, systems and compositions may use polynucleotide and polypeptide libraries, and polypeptide display approaches to develop the polypeptides of interest.

In an aspect, the present disclosure provides a high throughput method for identifying an optimized polypeptide, comprising: (a) providing a first library of polynucleotides encoding a first library of variantpolypeptides; (b) processing the first library of polynucleotides to produce the first library of variant polypeptides wherein the variant polypeptides are attached to the first library of polynucleotides; (c) identifying one or more characteristics comprising an equilibrium binding constant, a kinetic binding constant, a protein stability measurement, an enzyme activity, a fractional activity, a nonspecific binding potential, an aggregation potential, a hydrophobicity, a protein expression level, or a maturation time of at least a portion of the first library of variant polypeptides; (d) providing a second library of polynucleotides encoding a second library of variant polypeptides selected based at least on one or more characteristic identified in (c); (e) processing the second library of polynucleotides to produce the second library of variant polypeptides wherein the variant polypeptides are attached to the second library of polynucleotides; and (f) analyzing the second library of variant polypeptides to produce optimized data.

In another aspect, the present disclosure provides a high throughput method for measuring a characteristic of a polypeptide, comprising: (a) providing a first library of polynucleotides attached to a solid surface, wherein the library of polynucleotides encode a library of variant polypeptides; (b) processing the library of polynucleotides to produce the library of variant polypeptides, wherein the variant polypeptides are attached to the library of polynucleotides; and (c) identifying one or more of characteristics comprising an equilibrium binding constant, a kinetic binding constant, a protein stability measurement, an enzyme activity, a fractional activity, a nonspecific binding potential, an aggregation potential, a hydrophobicity, a protein expression level, or a maturation time of at least a portion of the library of variant polypeptides.

In another aspect, the present disclosure provides a high throughput method for screening a plurality of polypeptides, comprising: (a) providing a first library of polynucleotides encoding a library of variant polypeptides, wherein the first library of variant polypeptides comprises at least 90% of all single amino acid variants wherein amino acid residues are substituted for an amino acid selected from a set of twenty different amino acids; (b) processing the first library of polynucleotides to produce the first library of variant polypeptides wherein the variant polypeptides are attached to the first library of polynucleotides; and (c) identifying one or more characteristics of polypeptides of the first library of variant polypeptides.

In another aspect, the present disclosure provide a high throughput method for screening a plurality of polypeptides, comprising: (a) providing a first library of polynucleotides encoding a first library of variant polypeptides, wherein the first library of variant polypeptides comprises single amino acid variants polypeptides corresponding to at least 90% of possible single nucleotide variants for a given reference sequence in a reference polypeptide, wherein for a given single amino acid variant, the amino acid residue is substituted for another amino acid selected from a set of twenty different amino acids; (b) processing the first library of polynucleotides to produce the first library of variant polypeptides wherein the variant polypeptides are attached to the first library of polynucleotides; and (c) identifying one or more characteristics of polypeptides of the first library of variant polypeptides.

In some embodiments, the one or more characteristics comprises an equilibrium binding constant, a kinetic binding constant, a protein stability measurement, an enzyme activity, a fractional activity, a nonspecific binding potential, an aggregation potential, a hydrophobicity, a protein expression level, or a maturation time of at least a portion of the first library of variant polypeptides

In some embodiments, the method further comprises: (d) providing a second library of polynucleotides encoding a second library of variant polypeptides selected based at least on one or more characteristic identified in (c); (e) processing the second library of polynucleotides to produce the second library of variant polypeptides wherein the variant polypeptides are attached to the second library of polynucleotides; and (f) analyzing the second library of variant polypeptides to produce optimized data. In some embodiments, the method further comprises (g) identifying an optimized polypeptide based on the optimized data. In some embodiments, the high throughput method does not comprise a cell. In some embodiments, the first library of polynucleotides is a library of deoxyribonucleic acid molecules.

In some embodiments, the equilibrium binding constant is a dissociation constant (K_{d}). In some embodiments, the equilibrium binding constant is an association constant (Kₐ). In some embodiments, the kinetic binding constant is an association rate constant (kₒₙ). In some embodiments, the kinetic binding constant is a dissociation rate constant (k_{off}). In some embodiments, the protein stability measurement is a protein melting temperature (Tₘ). In some embodiments, the protein stability measurement is a midpoint denaturation concentration of a chemical denaturant (Cₘ).

In some embodiments, the method further comprises in (d), identifying negative variations, positive variations, and neutral variations from the first library of variant polypeptides. In some embodiments, the neutral variations have a dissociation constant greater than 0.25 times and less than 2 times a dissociation constant of a starting polypeptide. In some embodiments, the positive variations have a dissociation constant less than or equal to 0.25 times a dissociation constant of a starting polypeptide. In some embodiments, the negative variations have a dissociation constant greater than or equal to 2 times a dissociation constant of a starting polypeptide.

In some embodiments, the first library of variant polypeptides comprises single amino acid variants wherein amino acid residues are substituted for an amino acid selected from a set of amino acids. In some embodiments, the set of amino acid comprises 10 different amino acids. In some embodiments, the set of amino acid comprises 20 different amino acids. In some embodiments, the set of amino acids comprises alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. In some embodiments, the first library of variant polypeptides consists of variants of a starting polypeptide and the starting polypeptide. In some embodiments, the first library of variant polypeptides comprises double amino acid variants of interacting amino acid pairs. In some embodiments, the double amino acid variants of interacting amino acid pairs comprise variants wherein amino acid residues of the interacting amino acid pairs are substituted for all twenty amino acids. In some embodiments, the interacting amino acid pairs are identified by via a crystal structure of the original polypeptide. In some embodiments, the interacting amino acid pairs comprise inter-polypeptide interactions and intra-polypeptide interactions. In some embodiments, the first library of variant polypeptides comprises single amino acid insertions at each position. In some embodiments, the first library of variant polypeptides comprises single amino acid deletions. In some embodiments, the first library of variant polypeptides comprises double amino acid deletions. In some embodiments, the first library of variant polypeptides comprises triple amino acid deletions. In some embodiments, the first library of variant polypeptides comprises at least four amino acid deletions. In some embodiments, analyzing the first library of variant polypeptides comprises transcribing and translating a polynucleotide of the first library of variant polynucleotides, wherein the polypeptide encoded by the polynucleotide is attached to the polynucleotide. In some embodiments, identifying the equilibrium binding constant, kinetic binding constant, protein stability measurement, enzyme activity, fractional activity, nonspecific binding potential, aggregation potential, hydrophobicity, protein expression level, or maturation time comprises performing a binding assay on the first library of variant polypeptides. In some embodiments, the identifying the equilibrium binding constant, kinetic binding constant, protein stability measurement, enzyme activity, fractional activity, nonspecific binding potential, aggregation potential, hydrophobicity, protein expression level, or maturation time comprises sequencing the first library of polynucleotides and associating sequences of the first library of polynucleotides with the binding assay. In some embodiments, the binding assay comprises assaying binding of the first library of variant polypeptides to an antigen. In some embodiments, the binding assay comprises assaying binding of the first library of variant polypeptides to more than one antigen. In some embodiments, the binding assay comprises assaying binding of the first library of variant polypeptides to a plurality of antigens. In some embodiments, the method further comprises identifying a variant polypeptide that binds to two or more antigens of the plurality of antigens. In some embodiments, the further comprising identifying a variant polypeptide that binds to at least one antigen of the plurality of antigens and does not bind to a different antigen of the plurality of antigens. In some embodiments, the method further comprises identifying a variant polypeptide that does not bind to the plurality of antigens. In some embodiments, the identifying the equilibrium binding constant, kinetic binding constant, protein stability measurement, enzyme activity, fractional activity, nonspecific binding potential, aggregation potential, hydrophobicity, protein expression level, or maturation time comprises generating binding data for more than one target. In some embodiments, the second library is generated based at least on binding data for more than one target. In some embodiments, the processing the second library of variant polypeptides comprises transcribing and translating a polynucleotide of the second library of variant polynucleotides, wherein the polypeptide encoded by the polynucleotide is attached to the polynucleotide. In some embodiments, identifying the optimized polypeptide comprises performing a binding assay on the second library of variant polypeptides encoded by the second library of polynucleotides. In some embodiments, identifying the equilibrium binding constant, kinetic binding constant, protein stability measurement, enzyme activity, fractional activity, nonspecific binding potential, aggregation potential, hydrophobicity, protein expression level, or maturation time comprises sequencing the second library of polynucleotides and associating sequences of the second library of polynucleotides with the binding assay. In some embodiments, the second library of variant polypeptides comprises at least 10⁴ polypeptides. In some embodiments, the first library of polynucleotides comprises at least 10⁶ polynucleotides. In some embodiments, the first library of variant polypeptides comprises at least 10⁴ polypeptides. In some embodiments, the method is performed in less than 48 hours. In some embodiments, the first library of variant polypeptides comprises a library of individual VHH antibodies. In some embodiments, the second library of variant polypeptides comprises a library of VHH antibody fusions. In some embodiments, the first library of variant polypeptides comprises a library of individual single chain variable fragments (scFvs). In some embodiments, the second library of variant polypeptides comprises a library of individual single chain variable fragments (scFvs) fusions.

In another aspect, the present disclosure provides, a high throughput method for identifying an optimized polypeptide, comprising: (a) obtaining a dataset comprising binding data of an antigen to a first plurality of polypeptides and providing a plurality of polynucleotides based at least in part on the dataset; (b) providing a plurality of polynucleotides attached to a solid surface; (c) processing the plurality of polynucleotides to produce a second plurality of polypeptides; (d) exposing an antigen to the second plurality of polypeptides and detecting an interaction of at least one polypeptide of the second plurality of polypeptides with the antigen; (e) generating sequence data comprising (i) a sequence of at least the at least one polypeptide, or (ii) a sequence of the corresponding polynucleotide that encodes the at least one polypeptide; (f) based at least in part on sequence data and the detecting, generating a plurality of fusions polypeptides wherein a fusion polypeptide of the plurality of fusion polypeptides comprises a polypeptide from each of the first plurality of polypeptides or the second plurality of polypeptides capable of binding the antigen; and (g) repeating (a) through (e), wherein the dataset comprises binding data of an antigen to the plurality of polypeptide fusions to identify the optimized polypeptide.

In another aspect, the present disclosure provide a method for identifying an optimized polypeptide, comprising: (a) providing a plurality of polynucleotides attached to a solid surface wherein the plurality of polynucleotides encode a plurality of fusion polypeptides, wherein a fusion polypeptide of the plurality of fusion polypeptides comprises two or more domains; (b) processing the plurality of polynucleotides to produce a plurality of fusion polypeptides; (c) exposing an antigen to the plurality of fusion polypeptides and detecting an interaction of at least one fusion polypeptide of the plurality of fusion polypeptides with the antigen; (d) generating sequence data comprising (i) a sequence of at least the at least one fusion polypeptide, or (ii) a sequence of the corresponding polynucleotide that encodes the at least one fusion polypeptide; and (e) based at least in part on the sequence data, the detecting, and a dataset comprising binding data of an antigen to a plurality of single domain polypeptides, generating an optimized polypeptide capable of binding the antigen. In some embodiments, the dataset is generated by identifying an polypeptide of the first plurality of polypeptides that can interact with the antigen. In some embodiments, the dataset is generated at least by exposing the antigen to the first plurality of polypeptides and detecting an interaction of at least one polypeptide of the first plurality of polypeptides with the antigen. In some embodiments, the first plurality of polypeptides is generated by (i) providing a plurality of first polynucleotides encoding a plurality of first polypeptides; (ii) providing a plurality of first capture probes attached to a solid surface configured to anneal to the first plurality of polynucleotides to produce a plurality of captured polynucleotides; (iii) processing the plurality of captured polynucleotides to produce the first plurality of polypeptides. In some embodiments, the data pertaining to first plurality of polypeptides comprises sequence data generated at least by sequencing the plurality of captured polynucleotides, wherein the plurality of capture polynucleotides is a plurality of VHH polynucleotides.

In some embodiments, the interaction of at least one polypeptide of the plurality of polypeptides with the antigen comprises identifying a quantitative characteristic of the polypeptide. In some embodiments, identifying the quantitative characteristic of the polypeptide further comprises identifying the polypeptide as comprising one or more of a negative, neutral or positive mutation. In some embodiments, the plurality of fusion polypeptides comprises a polypeptide for at least 50%, 60%, 70%, 80%, 90%, or more, of all possible fusion pair combinations or permutations of the polypeptides of the first plurality of polypeptides. In some embodiments, the plurality of fusion polypeptides comprises a polypeptide for of all possible fusion pair combinations or permutations of the polypeptides of the first plurality of polypeptides. In some embodiments, the dataset comprises data corresponding to single domain polypeptides that correspond to one or domains of the fusion polypeptides. In some embodiments, the dataset is generated by identifying a single domain polypeptide that can interact with the antigen. In some embodiments, the dataset is generated at least by exposing the antigen to a plurality of single domain polypeptides and detecting an interaction of at least one single domain polypeptide of the plurality of single domain polypeptides with the antigen. In some embodiments, the plurality of single domain polypeptides is generated by (i) providing a plurality of single domain polynucleotides encoding a plurality of single domain polypeptides, wherein the single domain polynucleotides are coupled to a solid surface; (iii) processing the plurality of single domain polynucleotides to produce the plurality of single domain polynucleotides polypeptides. In some embodiments, the dataset comprises sequence data generated at least by sequencing the plurality of single domain polynucleotides. In some embodiments, the single domain polypeptide comprises a VHH. In some embodiments, the fusion polypeptide comprises a VHH-VHH fusion. In some embodiments, the plurality of fusion polypeptide comprise a sequence corresponding to one or more polypeptide of the plurality of single domain polypeptides. In some embodiments, a fusion polypeptide of the plurality of fusion peptides comprises sequences of two polypeptides of the plurality of single domain polypeptides. In some embodiments, the plurality of fusion polypeptides comprises a polypeptide for at least 50%, 60%, 70%, 80%, 90%, or more, of all possible fusion pair combinations or permutations of the single domain polypeptides of the plurality of single domain polypeptides. In some embodiments, the plurality of fusion polypeptides comprises a polypeptide for of all possible fusion pair combinations or permutations of the single domain polypeptides of the plurality of single domain polypeptides. In some embodiments, the plurality of single domain polypeptides comprises a plurality of single domain polypeptides differing by a single point mutation. In some embodiments, the plurality of single domain polypeptides comprises a plurality of single domain polypeptides differing by a single point mutation in a binding interface. In some embodiments, the plurality of single domain polypeptides comprises a plurality of single domain antibody fragments differing by a single point mutation in a CDR. In some embodiments, the plurality of single domain polypeptides comprises a plurality of 20 polypeptides wherein a different amino acid is encoded at a given residue.

In some embodiments, detecting the interaction of at least one single domain polypeptide of the plurality of single domain polypeptides with the antigen comprises identifying a quantitative characteristic of the single domain polypeptide. In some embodiments, the identifying the quantitative characteristic of the polypeptide further comprises identifying the single domain polypeptide as comprising one or more of a negative, neutral or positive mutation. In some embodiments, the detecting the interaction of at least one fusion polypeptide of the plurality of fusion polypeptides with the antigen comprises identifying a quantitative characteristic of the fusion polypeptide. In some embodiments, identifying the quantitative characteristic of the polypeptide further comprises identifying the fusion polypeptide as comprising a bi-epitopic interaction. In some embodiments, the identifying the fusion polypeptide as comprising an avidity-enhanced interaction comprises comparing the quantitative characteristic of the fusion polypeptide with quantitative characteristics of a first single domain or a second single domain, wherein the sequence of the fusion polypeptide comprises the sequence of the first single domain and the second single domain. In some embodiments, the avidity-enhanced interaction is identified when the quantitative characteristic of the fusion polypeptide is greater than the quantitative characteristics of the first single domain or the second single domain. In some embodiments, the optimized polypeptide comprises additional mutations of the fusion polypeptide identified as comprising an avidity-enhanced interaction, wherein the mutation increases the binding affinity of the fusion polypeptide to the antigen. In some embodiments, the data comprising binding data of an antigen to a plurality of the single domain polypeptides is obtained at a same time as (c) or (d) is performed. In some embodiments, the data comprising binding data of an antigen to a plurality of the single domain polypeptides is obtained prior to (a), and wherein the providing the plurality of polynucleotides attached to a solid support is based at least in part on the dataset.

In some embodiments, the plurality of fusion polypeptides comprise sequences of single domain polypeptides comprising a moderate affinity to the antigen. In some embodiments, the plurality of fusion polypeptides comprise sequences of single domain polypeptides comprising minimal affinity or no affinity to the antigen. In some embodiments, the sequences of single domain polypeptides comprising minimal affinity or no affinity comprise a substantially similar size or length to a single domain polypeptide that is capable of binding the antigen. In some embodiments, the sequences of single domain polypeptides comprising minimal affinity or no affinity comprise no more than a 10% difference in size or length to a single domain polypeptide that is capable of binding the antigen. In some embodiments, a single domain polypeptide of the plurality of single domain polypeptides comprises a N-terminal linker or a C-terminal spacer. In some embodiments, a single domain polypeptide of the plurality of single domain polypeptides comprises a N-terminal linker and a C-terminal spacer. In some embodiments, the plurality of single domain polypeptides comprises a plurality of different N-terminal linker sequences and different C-terminal spacer sequences. In some embodiments, the dataset is derived from data in a public database.

In some embodiments, the fusion polypeptide is a polypeptide-Fc fusion. In some embodiments, the polypeptide-Fc fusion comprises an antibody fragment crystallization region (Fc region) capable of binding the antigen. In some embodiments, the fusion polypeptide comprises a chimeric antigen receptor. In some embodiments, the fusion polypeptide comprises a VHH nanobody. In some embodiments, the fusion polypeptide comprises a pair of bivalent VHH nanobodies. In some embodiments, the fusion polypeptide comprises a pair of bi-epitopic VHH nanobodies. In some embodiments, the fusion polypeptide comprises multivalent VHH nanobodies. In some embodiments, the fusion polypeptide comprises a linker connecting a first domain of the fusion polypeptide and a second domain of the fusion polypeptide. In some embodiments, the first domain comprises a VHH. In some embodiments, the second domain comprises a VHH. In some embodiments, the first domain comprises a first VHH and the second domain comprise a second VHH. In some embodiments, the first VHH and the second VHH bind a same antigen. In some embodiments, the same antigen comprises a polypeptide, lipid, or carbohydrate, or cell. In some embodiments, the linker comprises at least 12 amino acids. In some embodiments, the linker comprises at least 20 amino acids. In some embodiments, the linker comprises at least 30 amino acids. In some embodiments, the linker comprises a net positive charge. In some embodiments, the linker comprises a net negative charge. In some embodiments, the linker comprises a net neutral charge.

In some embodiments, the plurality of polynucleotides comprises at least 10⁴ polynucleotides. In some embodiments, the optimized polypeptide comprise an increased avidity effect. In some embodiments, the prior to (a) the solid surface comprises plurality of capture oligonucleotides configured to anneal to a plurality of precursor polynucleotides, and wherein the plurality of precursor polynucleotides anneal to the plurality of capture nucleotide thereby producing the plurality of polynucleotides attached to a solid surface. In some embodiments, the producing the plurality of polynucleotides attached to a solid surface comprises an amplification or extension of the plurality of precursor polynucleotides. In some embodiments, the amplification comprises bridge amplification. In some embodiments, the solid support comprises a bead. In some embodiments, the solid support comprises sequencing flow cell.

In some embodiments, (d) comprises sequencing the plurality of polynucleotides. In some embodiments, (e) comprises generating the optimized polypeptide based at least in part on the sequence data generated from of the sequencing of the plurality of polynucleotides and the detecting. In some embodiments, a fusion polypeptide of the plurality of fusion polypeptides comprises a N-terminal linker or a C-terminal spacer. In some embodiments, a fusion polypeptide of the plurality of fusion polypeptides comprises a N-terminal linker and a C-terminal spacer. In some embodiments, the a fusion polypeptide comprises a plurality of different N-terminal linker sequences and different C-terminal spacer sequences. In some embodiments, the optimized polypeptide comprises a bi-epitopic polypeptide. In some embodiments, the optimized polypeptide comprises a tri-epitopic polypeptide. In some embodiments, the optimized polypeptide comprises a tetra-epitopic polypeptide. In some embodiments, the optimized polypeptide comprises a multimeric polypeptide. In some embodiments, the optimized polypeptide comprises at two or more domains capable of binding to the antigen, wherein at least two domains are identical. In some embodiments, the optimized polypeptide comprises two or more domains capable of binding to the antigen, wherein the two or more domains are different from one another.

In another aspect, the present disclosure provides a method for identifying a bi-epitopic polypeptide, comprising: (a) providing a plurality of polynucleotides attached to a solid surface, wherein the plurality of polynucleotides encoding a plurality of VHH polypeptides; (b) processing the plurality of polynucleotides to produce the plurality of VHH polypeptides; (c) exposing an antigen to the plurality of polypeptides and detecting an interaction of at least one VHH polypeptide of the plurality of VHH polypeptides with the antigen; (d) sequencing the plurality of polynucleotides; (e) providing a second plurality of polynucleotides attached to a solid surface, wherein the second plurality of polynucleotides encode a plurality of VHH-VHH fusion polypeptides; (f) processing the plurality of second polynucleotides to produce a plurality of VHH-VHH fusion polypeptides; (g) exposing an antigen to the plurality of VHH-VHH fusion polypeptides and detecting an interaction of at least one VHH-VHH fusion polypeptide of the plurality of VHH-VHH fusion polypeptides with the antigen; (h) sequencing the second plurality of polynucleotides; and (i) based at least in part on sequence data generated from of the sequencing of (d) and (e) and the detecting of (c) and (g), generating a bi-epitopic polypeptide capable of binding the antigen.

In another aspect, the present disclosure provides a method for generating an optimized polypeptide comprising: (a) providing a plurality of polypeptides displayed on a solid substrate, wherein a polypeptide of the plurality of polypeptides comprises a binding domain, and one or more of a (i) N-terminal spacer, (ii) a C-terminal spacer, wherein the plurality of polypeptides comprises polypeptides comprising different combinations of N-terminal spacer sequences and C-terminal spacer sequences; (b) observing a signal of least two polypeptides of the plurality of polypeptides, wherein the signal corresponds to (i) a binding interaction of a polypeptide and an antigen or (ii) a physical characteristic of a polypeptide; (c) comparing the signals of the at least two polypeptide and determining the combination of N-terminal spacer sequences and C-terminal spacer sequences that generates a target signal.

In some embodiments, the N-terminal spacer or C-terminal spacer does not bind to the antigen. In some embodiments, the target signal comprises a signal below a threshold level. In some embodiments, the target signal comprises a signal above a threshold level. In some embodiments, the target signal comprises a highest signal of signals of the plurality of polypeptides. In some embodiments, the target signal comprises a lowest signal of signals of the plurality of polypeptides.

In some embodiments, the signal corresponds to an equilibrium binding constant, kinetic binding constant, protein stability measurement, enzyme activity, fractional activity, nonspecific binding potential, aggregation potential, hydrophobicity, protein expression level, or maturation time of a polypeptide.

In another aspect, the present disclosure provides a method for discovery of improved pairs of binders comprising: (a) providing a comprehensive dataset comprising (i) measured quantitative binding characteristics for a plurality of polypeptides comprising two domains, wherein the two domains are independently selected from a set of monomeric domains, wherein the plurality of polypeptides comprise all possible pairs of monomeric polypeptides; and (ii) measured quantitative binding characteristics of each monomeric domain of the set of monomeric domains as an individual monomer polypeptide; (b) comparing values of (i) and (ii) to identify polypeptides comprising improved pairs of binders that exhibit quantitative binding characteristics significantly greater than the binding characteristics of either component individual monomer polypeptide. In some embodiments, the improved pairs of binders are bi-epitopic binders. In some embodiments, the comprehensive data set comprises measured quantitative binding characteristics for set of individuals monomer polypeptides and measured quantitative binding characteristics for at least 50%, 60%, 70%, 80%, 90%, or more, of all possible tandem pair combinations of the set of individual monomer polypeptides. In some embodiments, the comprehensive data set comprises measured quantitative binding characteristics for set of individuals monomer polypeptides and measured quantitative binding characteristics for all possible tandem pair combinations of the set of individual monomer polypeptides.

In another aspect, the present disclosure provides a high throughput method for identifying affinity—and avidity—optimized tandem polypeptides, comprising: (a) providing a first library of polynucleotides encoding a first library of monomeric variantpolypeptides; (b) processing the first library of polynucleotides to produce the first library of variant polypeptides wherein the variant polypeptides are attached to the first library of polynucleotides; (c) analyzing the first library of variant polypeptides to produce data; (d) identifying the binding affinity of at least a portion of the first library of variant polypeptides based on the data; (e) providing a second library of second polynucleotides encoding a second library of monomeric variant polypeptides from the first library based on the binding data from the first library; (f) providing a third library of polynucleotides encoding a plurality of tandem polypeptides comprising different combinations of the monomeric variant polypeptides corresponding to the first library, wherein a tandem polypeptide of the plurality of tandem polypeptide comprises a first monomeric variant polypeptide and a second monomeric variant polypeptide. (g) processing the second and third libraries of polynucleotides to produce the second and third libraries of variant polypeptides wherein the variant polypeptides are attached to the second and third library of polynucleotides; (h) analyzing the second and third libraries of variant polypeptides to identify affinity enhancing monomer polypeptide variants and avidity-enhancing tandem polypeptides; and (i) combining avidity and affinity enhancements identified in the second and third libraries by substituting the individually optimized monomers identified in the second library into the corresponding positions in the avidity-enhancing tandem pairs discovered from the second library. In some embodiments, the third library comprises a plurality of polypeptides comprising a different linker between the first monomeric variant polypeptide and the second monomeric variant polypeptide. In some embodiments, the third library comprises monomeric variants polypeptides comprising a reduced affinity compared to a reference polypeptide based on the binding data from the first library.

In another aspect, the present disclosure provides composition comprising: an array of polypeptides displayed on a solid surfaces, wherein each polypeptide is co-localized to a corresponding polynucleotide that encode the polypeptide, wherein a polypeptide of the plurality of polypeptides comprises a first domain and a second domain, wherein the first domain and second domain are linked via a linker, wherein the first domain binds a first epitope and the second domain binds a second epitope, wherein the first epitope and second epitope are different. The composition may comprise array of polypeptides comprising polypeptide libraries as described elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. “The patent or application file contains at least one drawing executed in color. Copies of this patent or patent application publication with color drawing(s) will be provided by the Office upon request and payment of the necessary fee. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1A shows a schematic of a nanobody sequence for initial display selection. FIG. 1B shows a representation of the nanobody library displayed using ribosome display.

FIG. 2 shows a schematic of a method of disclosure wherein a DNA library is generated and quantified.

FIG. 3 shows a heat map of single mutation in CDR regions.

FIG. 4 shows a schematic of a method of disclosure wherein a DNA library is generated and quantified, followed by generation and quantification of a new library based on the analysis of a prior library.

FIG. 5 shows data relating to polypeptides generated by the methods of the disclosure.

FIG. 6 shows data relating to select polypeptides generated by the methods of the disclosure.

FIG. 7 shows a schematic of polypeptides that may be generated using the methods of the disclosure.

FIG. 8 shows a schematic of multi-specific or selective polypeptides.

FIG. 9 shows a workflow schematic for generation of bi-epitopic polypeptides.

FIG. 10 shows heat maps of binding data for single mutants in the CDR regions of representative VHHs in the dataset.

FIG. 11 shows a schematic of the design of a DNA library encoding tandem VHHs that may be expressed on chip, assayed for binding, and analyzed to find avidity enhancement using the methods of the disclosure.

FIG. 12A shows avidity enhancement data generated for a specific tandem VHH pair using the methods of the disclosure. FIG. 12B shows a heat map of avidity enhancement for all tandem VHH pairs in the experiment in both orientations.

FIG. 13A shows a distribution of the number of mutations in the VHH affinity optimization library generated using the methods of the disclosure FIG. 13B shows data relating to the affinity optimized VHHs generated to two distinct targets using the methods of the disclosure.

FIG. 14 shows a workflow schematic for generation of affinity optimized, avidity-enhanced multivalent tandem VHH pairs.

FIG. 15A-15C shows workflow schematics of (15A) sequential (“two-step”) optimization, (15B) discovery of tandem polypeptide pairs with enhanced avidity, and (15C) a combined workflow for discovery of affinity-optimized molecules formatted in tandem configurations with high avidity using the methods of the disclosure.

FIG. 16 shows a computer control system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The present disclosure provides methods, systems, and compositions for generation of polypeptide libraries and methods, systems, and compositions for displaying the libraries to identify or determine characteristics of the polypeptides. Approaches described herein may be effective for the optimization or the generation of polypeptides with particular characteristics. Specifically, approaches may be used to generate antibodies or antibody fragments that are able to bind antigens at low concentrations. The methods described herein may allow for highly multiplexed quantitative assays which may result in the generation of data that would otherwise be difficult to obtain quickly. This data may be leveraged and used to guide the subsequent iterations of the method described, or have combined with other data generated to create polypeptide that may be optimized to have multiple characteristics. The methods may be iteratively performed by using data gathered by an earlier iteration to guide the construction of later iterations to quickly and efficiently identify polypeptides with extreme or rare functionality. The generation of large data sets may be a leveraged to construct polypeptides that other methods, such as directed evolution, would be unable to identify. Because of the size of sequence space that one may need to analyze to identify polypeptides of interest, there is a need to analyze a large amount of potential polypeptides and generate quantitative data in a fast, tunable, and customizable manner.

### Polypeptide Library Construction

In various aspect of the disclosure, a polypeptide library is constructed. In order to identify and generate polypeptides with particular properties of interest, polypeptide libraries may be constructed based on sets of parameters. Using polypeptide library display methods as described elsewhere herein, the polypeptide library may be subjected to analysis.

In some embodiments, the polypeptide library comprises a wild type or reference polypeptide. In some embodiments, the polypeptide library may comprise a variant of a wild type or reference polypeptide. The variant may comprise a substitution mutation, an insertion, or a deletion. Polypeptide libraries may comprise polypeptide variants with mutations at 1, 2, 3, 4, 5, 6, 7, 8, 9, 10,11, 12, 13, 14, 15, 16, 17, 18, 19, 20, 30, 40, 50, 60,70, 80, 90, 100 or more amino acids. The polypeptide library may comprise polypeptides corresponding to all possible single point substitution variants for a single residue. The single point mutation may comprise substituting an amino acid for another amino acid selected from a set of amino acids. The set of amino acids may be 1, 2, 3, 4, 5, 6, 7, 8, 9, 10, 11, 12, 13, 14, 15, 16, 17, 18, 19, 20, 21, 22, 23, 24, 25, or more amino acids. The set of amino acids may comprise alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. The set of amino acids may comprise alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, or combinations thereof. For example, the polypeptide library may comprise 20 polypeptides (e.g. based on the 20 canonical amino acids), wherein at a first residue the amino acid is a different amino acid, and all other amino acids are the same. In this way, the polypeptide library may be analyzed to generate data relating to how an amino acid at a particular residue number may affect the properties of a polypeptide. The polypeptide library may comprise polypeptides corresponding to single point substitutions for 20 amino acids at all residues in the polypeptide. For example, for a 100 amino acid long polypeptide, for each residue 20 variants are generated corresponding to each canonical amino acid, resulting in 2,000 (20×100) different polypeptides. Using this approach, a polypeptide library may be analyzed to generate data relating to, for the entire length of a polypeptide, how an amino acid at a particular residue number may affect the properties of a polypeptide.

The polypeptide library may comprise polypeptides corresponding to single point substitutions for 20 amino acids at all residues in a region of the polypeptide. For example, a particular domain of the polypeptide may be correlated to a function, such as binding to an antigen or other target. The polypeptide library may comprise polypeptides corresponding to single point substitutions for 20 amino acids at residues specific to the particular domain. For example, the polypeptide may be an antibody, or fragment of the antibody and the particular domain may be a complementarity determining region (CDR). The polypeptide library may comprise polypeptides corresponding to at least 80% of all single point substitutions for 20 amino acids at all residues in a region of the polypeptide. The polypeptide library may comprise polypeptides corresponding to at least 90% of all single point substitutions for 20 amino acids at all residues in a region of the polypeptide. The polypeptide library may comprise polypeptides corresponding to at least 95% of all single point substitutions for 20 amino acids at all residues in a region of the polypeptide. The polypeptide library may comprise polypeptides corresponding to at least 99% of all single point substitutions for 20 amino acids at all residues in a region of the polypeptide. The polypeptide library may comprise polypeptides corresponding to at least 80% of all single point substitutions for 20 amino acids at all residues in the polypeptide. The polypeptide library may comprise polypeptides corresponding to at least 90% of all single point substitutions for 20 amino acids at all residues in the polypeptide. The polypeptide library may comprise polypeptides corresponding to at least 95% of all single point substitutions for 20 amino acids at all residues in the polypeptide. The polypeptide library may comprise polypeptides corresponding to at least 99% of all single point substitutions for 20 amino acids at all residues in the polypeptide. The amino acids may comprise alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine.

Polypeptide libraries may be constructed based at least on structural data. A structure of a reference (or variant) polypeptide may be generated or may have been generated previously. A structure may be generated based on structure determination methods, for example x-ray crystallography or nuclear magnetic resonance (NMR) spectroscopy, or other methods for elucidating structural information. Using the structural data of the polypeptide, residues may be identified as interacting with other residues. Polypeptides of the polypeptide library may be generated based on information relating to the interaction of residues according to a structural model. For example, a reference polypeptide model may show an interaction between a residue A and a residue B. The polypeptide library may comprise a double variant in which residue A and residue B are variants as compared to a reference or wild type polypeptide. This may be such that for each variant amino acid at residue A, all possible amino acid variants at residue B are generated, and vice versa. For a given residue A and residue B,400 polypeptides (20 possible amino acids at residue A×20 possible amino acids at residue B) may be generated. Using this approach, a polypeptide library may be analyzed to generate data relating to how interacting amino acids at particular residue numbers may affect the properties of a polypeptide.

Polypeptides of the polypeptide library may also correspond to deletions of amino acids as compared to a wildtype or reference polypeptide. A polypeptide may comprise a deletion variant wherein any single amino acid or groups of amino acids have been deleted. The polypeptide may comprise deletions of 1, 2, 3, 4, 5, 6, 7, 8, 9, 10,11, 12, 13, 14, 15, 16, 17, 18, 19, 20, 30, 40, 50, 60,70, 80, 90, 100 or more amino acids. The polypeptide may comprise deletions of 1, 2, 3, 4, 5, 6, 7, 8, 9, 10,11, 12, 13, 14, 15, 16, 17, 18, 19, 20, 30, 40, 50,60,70, 80, 90, 100, or more contiguous amino acids. The deletion may be located at any part of the polypeptide chain.

Polypeptides of the polypeptide library may also correspond to insertions of amino acids as compared to a wildtype or reference polypeptide. A polypeptide may comprise a insertion variant wherein any single amino acid or groups of amino acids have been inserted. The polypeptide may comprise insertions of 1, 2, 3, 4, 5, 6, 7, 8, 9, 10,11, 12, 13, 14, 15, 16, 17, 18, 19, 20, 30, 40, 50, 60,70, 80, 90, 100, or more amino acids. The polypeptide may comprise insertions of 1, 2, 3, 4, 5, 6, 7, 8, 9, 10,11, 12, 13, 14, 15, 16, 17, 18, 19, 20, 30, 40, 50, 60,70, 80, 90, 100, or more contiguous amino acids. The insertion may be located at any part of the polypeptide chain.

A polypeptide library may comprise combinations of polypeptide libraries as described elsewhere herein. For example, the polypeptide library may comprise polypeptides comprising insertion variants and polypeptides with single point substitution variants.

A polypeptide library may be generated based on data generated from polypeptide libraries as described elsewhere herein. For example, a first polypeptide library may be generated corresponding to single point substitutions across a particular domain of the polypeptide. The polypeptide library may be subjected to an assay wherein binding to a particular antigen is analyzed. Data corresponding to the binding of polypeptides in the library may demonstrate that certain single point substitution variants may increase or decrease binding, or remain the same, as compared to a reference or wild type polypeptide. Using the data, polypeptides comprising multiple single point substitution variants may be generated. For example, data on a polypeptide may indicate that: (1) a single point variant of residue A to an amino acid X may increase binding; and (2) a single point variant of residue B to an amino acid Y may increase binding. A polypeptide may be generated for a polypeptide library comprising a first singe point variant of residue A to an amino acid X, and a second single point variant of residue B to an amino acid Y, and assayed. Synergistic effects of variants may be analyzed and allow for the generation of polypeptides with improved characteristics. Polypeptide libraries may comprise polypeptides comprising combinations of variants that were determined to improve or maintain a characteristic of the polypeptide. For example, 10 variants may be shown to have improved or neutral binding to an antigen. Polypeptide libraries comprising combinations of the 10 variants may be generated wherein a first polypeptide may have any 2 variants of the 10 possible variants, a second polypeptide may have any 3 variants of the 10 possible variants, and so on.

These library construction approaches may be used iteratively and generate a multi-step/multi-library approach to optimizing or generating polypeptides comprising a particular characteristic. A first library may be generated and assayed to determine characteristics of polypeptides of the first polypeptide library. Using the data generated, a second polypeptide library may be constructed that takes in account the data, for example how a variant affects a characteristic. The second library may be assayed and data may be generated to identify a polypeptide with a particular characteristic. This may be repeated, for example, wherein a third library is generated based on data generated from second library, or wherein a nth+1 library is generated from data generated from a nth library (or other library). Additionally, the data for a library may be analyzed by an algorithm or used as training sets for a predictive algorithm or machine learning, such to identify variants of interest for use in a next library.

Libraries may be constructed from sequences analyzed in previously generated libraries or from other data sources. For example, libraries may be generated that combine polypeptides that were analyzed in a previously generated library. A first library may be generated that comprises a plurality of polypeptides that bind to a given antigen. A second library may use one or more sequences of the plurality of polypeptides from the first library in combination with another sequence of the plurality of polypeptides from the first library. A first library may comprise plurality of different scaffolds that comprises a characteristic. A second library may comprise a plurality of fusions of the different scaffolds that were analyzed in the first library. A first library may comprise a plurality of binding polypeptides comprising different structures or point mutations. A second library may comprise bi-valent or bi-epitopic polypeptides comprising a combination of binding polypeptides from the first library. A second library may comprise bi-valent or bi-epitopic polypeptides comprising all combinations of binding polypeptides from the first library. A second library may comprise bi-valent or bi-epitopic polypeptides comprising all permutations of binding polypeptides from the first library.

Libraries of polypeptides may be generated from a corresponding library of polynucleotides. The libraries may comprise at least 10³, 10⁴, 10⁵, 10⁶, 10⁷, 10⁸, 10⁹, or more polynucleotides. The libraries may comprise 10³, 10⁴, 10⁵, 10⁶, 10⁷, 10⁸, 10⁹, or more polypeptides. The libraries may comprise at least 10³, 10⁴, 10⁵, 10⁶, 10⁷, 10⁸, 10⁹, or more polynucleotides on a single substrate, sequencing chip, or in a sample volume. The libraries may comprise at least 10³, 10⁴, 10⁵, 10⁶, 10⁷, 10⁸, 10⁹, or more polypeptides on a single substrate, sequencing chip, or in a sample volume.

A polypeptide may be any polymer composed of amino acids. The polypeptide may bind to another molecule, perform a reaction (physical or chemical), transduce a signal, act as a structural component, generate a movement, or other function. The polypeptide may be an antibody or a fragment (or fragments) of an antibody. For example, polypeptide may be a single chain variable fragment (scFv) or a nanobody (e.g. VHH).

The methods described in this disclosure may be used to identify or generate polypeptides comprising particular or improved characteristics. The methods described may be performed on any reference or wild type sequence to generate libraries of polypeptides. The methods may allow any reference polypeptide with a function to be optimized to have an improved function. The particular characteristic may be a stability of a polypeptide. The particular characteristic may be an enzymatic rate or other reaction parameters. The particular characteristic may comprise at least a particular binding affinity to a molecule or a dissociation constant. For example, with the methods described, an antibody or antibody fragment may be generated that has a high affinity to a target. A polypeptide generated may comprise a binding affinity to an antigen or target of at less than 1 nM. A polypeptide generated may comprise a binding affinity to an antigen or target of at no more than 100 nM, 10 nM, 1 nM, 100 pM, 10 pM, 1 pM or less.

The polypeptide generated may have an improved measured binding affinity compared to a reference or wild-type polypeptide. For example, the measured binding affinity may comprise a 10% improvement compared to a reference or wild-type polypeptide. For example, the measured binding affinity may comprise a 25% improvement compared to a reference or wild-type polypeptide. For example, the measured binding affinity may comprise a 50% improvement compared to a reference or wild-type polypeptide. For example, the measured binding affinity may comprise a 75% improvement compared to a reference or wild-type polypeptide. For example, the measured binding affinity may comprise a 100% improvement compared to a reference or wild-type polypeptide. For example, the measured binding affinity may comprise a 200% improvement compared to a reference or wild-type polypeptide. For example, the measured binding affinity may comprise a 300% improvement compared to a reference or wild-type polypeptide. For example, the measured binding affinity may comprise a 400% improvement compared to a reference or wild-type polypeptide. For example, the measured binding affinity may comprise a 500% improvement compared to a reference or wild-type polypeptide. For example, the measured binding affinity may comprise a 1,000% improvement compared to a reference or wild-type polypeptide. For example, the measured binding affinity may comprise a 100 fold improvement compared to a reference or wild-type polypeptide. For example, the measured binding affinity may comprise a 1000 fold improvement compared to a reference or wild-type polypeptide. For example, the measured binding affinity may comprise a 10,000 fold improvement compared to a reference or wild-type polypeptide. For example, the measured binding affinity may comprise a 100,000 fold improvement compared to a reference or wild-type polypeptide. For example, the measured binding affinity may comprise a 1,000,000 fold improvement compared to a reference or wild-type polypeptide. The generated polypeptide may be an avidity-enhanced polypeptide.

Avidity generally refers to the accumulated strength of multiple separate non-covalent interactions between a binding molecule and an antigen, and results in an increase in the measured binding affinity. An avidity effect may cause an increase in local concentration (of antigen or binding molecule) by having multiple antigen binding sites interact with an antigen. Whereas a single binding interaction may be broken and allow an antigen to be released and no longer interact with a binding molecule, a molecule with multiple binding site s (and multiple separate non-covalent interactions) may keep antigen bound even if an individual binding interaction is broken. An avidity-enhanced polypeptide may have multiple different binding interactions, such as a bi-epitopic binder which is able to bind two different epitopes. Similarly, a mono-epitopic multimeric binder may keep an antigen bound by “trading” the antigen between binding sites, and may effectively increase the local concentration of the binding sites, thereby increasing the measured binding affinity.

### Polypeptide Library Display

In various aspect of the disclosure, polypeptides are generated and displayed as library. Methods of displaying the polypeptide library may incorporate methods that can correlate a genotype and a corresponding phenotype. One such method for peptide display may comprises ribosome based display methods. Methods of display using ribosomes include methods described in US Pat. Appl. Pub. No. US2020/0048629 and U.S. Pat. No. 10,011,830, herein incorporated by reference. The methods of display may comprise the polypeptides displayed as a ribosomal translation product (e.g., a protein or peptide, a biologically active fragment thereof, or other ribosomally translated molecule) on a DNA template encoding it. The DNA template may comprise a promoter operably linked to an open reading frame (ORF). The DNA template may further comprise a molecular roadblock that blocks progress of an RNA polymerase during transcription of the DNA template. The molecular roadblock may cause the RNA polymerase to stall during transcription, such that the DNA template and transcribed mRNA remain associated. During translation of the RNA transcript, the stalled RNA polymerase at the molecular roadblock may block ribosomes from continuing translation, such that the ribosomes display the nascent peptide chain (e.g., protein or peptide, biologically active fragment thereof, or other ribosomally translated molecule) while remaining associated with the RNA transcript. If desired, the single-stranded mRNA, produced by transcription of the DNA template, may be cleaved proximal to the ribosome after the ribosome reaches the molecular roadblock.

The molecular roadblock may comprise a configuration of one or more molecules downstream of a transcribable region of DNA positioned such that when the RNA polymerase in the process of transcription encounters the roadblock, the polymerase stalls, forming a stable complex comprising the RNA polymerase, DNA template, and nascent RNA transcript. The roadblock may be a molecular entity, associated covalently or non-covalently with the DNA, or a chemical modification to the DNA, such as a chemical crosslink between strands of DNA that causes the RNA polymerase to stall. The roadblock can be placed at the 5′ end of the antisense DNA strand or the 3′ end of the sense DNA strand, or both. The roadblock may also include a molecule that binds selectively to a particular sequence of DNA at the appropriate location. In one embodiment, the molecular roadblock is formed by biotinylating the DNA either at the 3′ end of the sense strand or the 5′ end of the anti-sense strand, followed by binding of streptavidin, wherein the biotin-streptavidin complex serves as a molecular roadblock that blocks the RNA polymerase.

In addition, the DNA template may encode a mRNA having a ribosome stall sequence. In certain embodiments, the ribosome stall sequence comprises a stop codon (e.g., UAG (amber), UAA (ochre), or UGA (opal or umber) in the mRNA). In another embodiment, the ribosome stall sequence further comprises a polyproline-coding sequence adjacent to the stop codon. In one embodiment, the polyproline-coding sequence comprises a coding sequence for a triple-proline motif, wherein the coding sequence for the triple-proline motif is located before (i.e., on the 5′ side of) the stop codon. In another embodiment, the ribosome stall sequence further comprises an arginine-histidine-arginine coding sequence adjacent to the polyproline-coding sequence (e.g., triple-proline motif), wherein the arginine-histidine-arginine coding sequence is located before (i.e., on the 5′ side of) the polyproline-coding sequence. The ribosomal display methods may also be performed at conditions that cause the ribosome to stall. For example, amino acid starvation of the ribosome may be used. Amino acid Starvation may be achieved by limiting the amount of a particular amino acid (or tRNA or other associated reagent) such that the ribosome is unable to add the next amino acid in to the growing nascent peptide, thereby stalling the ribosome.

The mRNA may further comprise a Shine Dalgarno sequence. The Shine Dalgarno sequence may be optimized for a particular ORF of interest to promote efficient ribosome binding and translation initiation.

Polynucleotides used in the present disclosure can be derived from any nucleic acid of known or unknown sequence, and can be, for example, a fragment of genomic DNA or cDNA. For example, polynucleotides can be derived from a primary nucleic acid sample that has been randomly fragmented. Polynucleotides can also be obtained from a primary RNA sample by reverse transcription into cDNA. Individual polynucleotides may contain a whole gene or part of a gene or cDNA derived from mRNA that encodes a protein or peptide, or a biologically active polypeptide or peptide fragment thereof. Additionally, polynucleotides may comprise recombinant engineered constructs. The polynucleotides may encode polypeptides described throughout this disclosure. For example, a polynucleotide may encode a nanobody or an scFv.

Protein translation may be carried out using an in vitro cell-free expression system. Translation can be performed in vitro using a crude lysate from any organism that provides all the components needed for translation, including, enzymes, tRNA and accessory factors (excluding release factors), amino acids and an energy supply (e.g., GTP). Cell-free expression systems derived from Escherichia coli, wheat germ, and rabbit reticulocytes are commonly used. E. coli-based systems provide higher yields, but eukaryotic-based systems are preferable for producing post-translationally modified proteins. Alternatively, artificial reconstituted cell-free systems may be used for protein production. For optimal protein production, the codon usage in the ORF of the DNA template may be optimized for expression in the particular cell-free expression system chosen for protein translation. In addition, labels or tags can be added to proteins to facilitate high-throughput screening. See, e.g., Katzen et al. (2005) Trends Biotechnol. 23:150-156; Jermutus et al. (1998) Curr. Opin. Biotechnol. 9:534-548; Nakano et al. (1998) Biotechnol. Adv. 16:367-384; Spirin (2002) Cell-Free Translation Systems, Springer; Spirin and Swartz (2007) Cell-free Protein Synthesis, Wiley-VCH; Kudlicki (2002) Cell-Free Protein Expression, Landes Bioscience; herein incorporated by reference in their entireties.

In certain embodiments, protein translation is carried out using an in vitro cell-free expression system lacking one or more release factors, such that the ribosome is not released from the stop codon on the mRNA. One or more of the release factors, including release factor 1 (RF1), release factor 2 (RF2), and release factor 3 (RF3) may be absent, or all the release factors may be absent in the in vitro cell-free expression system. The release factors that are absent may depend on the stop codon chosen for inclusion in the stall sequence. For example, RF1 normally mediates release of a ribosome from the RNA transcript at an amber codon. Accordingly, if an amber codon is included in the stall sequence, RF1 may be omitted from the in vitro cell-free expression system. On the other hand, RF2 normally mediates release of a ribosome from an RNA transcript at either an ochre or opal codon. Therefore, RF2 may be omitted from the in vitro cell-free expression system if an ochre or opal codonis included in the stall sequence. In some embodiments, protein translation is carried out using an in vitro cell-free expression system lacking any release factors. Additionally, ribosome recycling factor (RRF) may also be omitted from an in vitro cell-free expression system to prevent release of a stalled ribosome from a transcribed RNA molecule.

In some embodiments, one or more non-canonical amino acids are incorporated into the ribosomal translation product, such as, but not limited to, D-amino acids, beta amino acids, or N-substituted glycines (peptoids). Non-canonical amino acids can be introduced into a protein or peptide in either a residue-specific or site-specific fashion. See, e.g., Link et al. (2003) Curr. Opin. Biotechnol. 14(6):603-609; Johnson et al. (2010) Curr. Opin. Chem. Biol. 14(6):774-780; Zheng et al. (2012) Biotechnol J. 7(1):47-60; herein incorporated by reference.

In some embodiments, the methods of polypeptides display may comprise providing conditions that allow only one RNA polymerase to initiate transcription on a polynucleotide. For example, the DNA template may further comprise a stall sequence, wherein the first RNA polymerase to initiate transcription stalls at a position on the DNA template such that initiation of any other polymerase is blocked. Transcription is carried out under conditions of nucleotide starvation, wherein the RNA polymerase stalls at a particular position on the DNA template because the nucleotide needed for addition at that position is not provided (see. e.g., Greenleaf and Block (2006) Science 313(5788):801; herein incorporated by reference). After the RNA polymerase stalls, any unbound polymerases are removed, for example, by washing, and then the missing nucleotide needed to resume transcription is added to allow transcription to continue until the one remaining RNA polymerase bound to the DNA template stalls at the molecular roadblock. Alternatively, the unbound RNA polymerases may be inactivated (e.g., using heparin) rather than being removed to ensure that only one RNA polymerase remains bound to the DNA template.

In some embodiments, the methods of polypeptides display may further comprise providing conditions that allow only one ribosome to initiate translation on the RNA transcript. For example, translation can be carried out under conditions of amino acid starvation, wherein the ribosome stalls at a particular position on the RNA transcript because the amino acid needed for addition at that position is not provided. Then, any unbound ribosomes can be removed, for example, by washing, and the missing amino acid needed to resume translation can be added to allow translation to continue until the one bound ribosome reaches the ribosome stall sequence.

The ribosomal translation product may comprise one or more linkers or spacers, for example, to facilitate display on a ribosome, cloning, purification, or detection, or to improve solubility. Short flexible linkers or spacers having, e.g., 20 or fewer amino acids (i.e., 20, 19, 18, 17, 16, 15, 14, 13, 12, 11, 10, 9, 8, 7, 6, 5, 4, 3, 2, or 1) are useful for separating domains in fusion constructs. Examples include short peptide sequences such as poly-glycine linkers (Glyn where n=2, 3, 4, 5, 6, 7, 8, 9, 10 or more), histidine tags (Hisn where n=3, 4, 5, 6, 7, 8, 9, 10 or more), linkers composed of glycine and serine residues, soluble polypeptide linkers, GSAT, SEG, and Z-EGFR linkers. Longer linkers, having a defined tertiary structure, can be used to facilitate display of a protein or peptide on ribosomes. Such linkers include, but are not limited to, fragments of gene III of filamentous phage M13mp192, a portion of the helical region of tolA, the extended region of tonB from E. coli, and a segment of protein D (pD) from the capsid of Lambda phage (see e.g., Yang et al. (2008) PLoS One 3(5):e2092; herein incorporated by reference). Other suitable linker amino acid sequences will be apparent to those skilled in the art. (See e.g., Argos (1990) J. Mol. Biol. 211(4):943-958; Crasto et al. (2000) Protein Eng. 13:309-312; George et al. (2002) Protein Eng. 15:871-879; Arai et al. (2001) Protein Eng. 14:529-532; and the Registry of Standard Biological Parts (partsregistry.org/Protein_domains/Linker). The polypeptides may comprise an N-terminal linker. The N-terminal linker may comprise amino acid sequences at the N-terminus of a displayed polypeptide. The polypeptides may comprise a C-terminal spacer. The C-terminal spacer may comprise additional amino acids at the C-terminus of a polypeptide.

A plurality of polypeptides may be displayed simultaneously or on a same given substrate (e.g. a solid surface such as a sequencing chip). For example, this method can be used to display the collective proteins or peptides encoded by a genomic library for an organism or a cDNA library produced from RNA from an organism, or a selected subset of proteins or peptides of interest expressed by an organism, or engineered proteins or peptides. The DNA library used for display may be entirely or partially synthetic and may contain sequences optimized for the expression of a particular set of polypeptides. The plurality of DNA templates may be free in solution or immobilized on a solid support. Polypeptide libraries and approaches for the constructions of polypeptide libraries are described elsewhere herein and any number of polypeptides from such libraries may be displayed simultaneously or on a same surface.

In some embodiments, a plurality of polynucleotides is immobilized on a solid support. The solid support may comprise, for example, glass, quartz, silica, metal, ceramic, or plastic. Exemplary solid supports include a slide, a bead, a plate, a gel, a membrane, or the inner surface of a flow cell or microchannel. Each DNA template can be located at a known, predetermined position on the solid support such that the identity of each protein produced from the DNA template can be determined from its position on the solid support. Alternatively, DNA templates can be bound randomly to the support, wherein the identity of the protein produced from each DNA template can be determined by sequencing of the associated DNA template or characterization of the protein itself. Immobilization or coupling of polynucleotides to a bead and methods of display of polypeptides may be used, such as those disclosed in WO2022026458A1, herein incorporated by reference.

Nucleic acids may be covalently linked to polypeptides or solid surfaces, such as a bead. Additionally, the polypeptides may also be linked to the bead, for example, via direct conjugation to the bead or via conjugation to a nucleic acid that is attached to a bead. In some embodiments, conjugation of the polypeptide to the nucleic acid molecule is catalyzed by a linking enzyme. In some embodiments, the polypeptide is conjugated to the nucleic acid molecule by expressed protein ligation or by protein trans-splicing. In some embodiments, the polypeptide is conjugated to the nucleic acid molecule by formation of a leucine zipper. In some embodiments, the bead or the nucleic acid molecule is conjugated to a capture moiety and the polypeptide includes a linkage tag, wherein the capture moiety and the linkage tag are conjugated, thereby conjugating the bead to the polypeptide or conjugating the nucleic acid molecule to the polypeptide. The linking enzyme may be a sortase, a butelase, a trypsiligase, a peptiligase, a formylglycine generating enzyme, a transglutaminase, a tubulin tyrosine ligase, a phosphopantetheinyl transferase, a Spy Ligase, or a SnoopLigase.

Nucleic acids can be coupled to a solid support by physical or chemical means using any method known in the art. A substrate may be added to the surface of a solid support to facilitate attachment of DNA templates. DNA array fabrication methods are well-known, and include various photochemistry-based methods, laser writing, electrospray deposition, inkjet and microjet deposition or spotting technologies, photolithographic oligonucleotide synthesis processes, as well as contact printing technologies, including contact pin printing and microstamping. The combination of suitable robotics, micromechanics-based systems, and microscopical techniques makes technically feasible the ordered deposition of up to millions of nucleic acids per cm2 on a solid support. See e.g., Rehman et al. (1999) Nucleic Acids Research 27:649-655; Heller et al. (2002) Annu. Rev. Biomed. Eng. 4:129-153; Dufva (2009) Methods Mol. Biol. 529:1-22; Sethi et al. (2008) Bioconjug Chem. 19(11):2136-2143; Adessi et al. (2000) Nucleic Acids Res. 28(20):E87; Okamoto et al. (2000) Nat. Biotechnol. 18(4):438-441; Barbulovic-Nad et al. (2006) Crit. Rev. Biotechnol. 26(4):237-259; herein incorporated by reference.

In one embodiment, acrylamide-modified nucleic acids are immobilized on a solid support containing exposed acrylic groups (e.g., silanized glass or plastic). The acrylamide group can be added to a nucleic acid during oligonucleotide synthesis using an acrylamide phosphoramidite. The acrylamide modification copolymerizes with acrylamide monomers to allow formation of a stable polyacrylamide co-polymer containing the immobilized nucleic acid. A layer containing immobilized DNA can be fabricated on a support by polymerizing an acrylamide matrix on the surface of the support and adding acrylamide-modified nucleic acids. Polymerization is catalyzed using standard chemical or photochemical methods. See, e.g., Rehman et al. (1999) Nucleic Acids Research 27:649-655; herein incorporated by reference in its entirety.

A polynucleotide can be immobilized on a solid support by hybridization to a complementary capture oligonucleotide attached to the surface of the solid support. A capture oligonucleotide may have a unique sequence complementary to a single DNA template in a mixture of DNA templates to allow selective capture of a particular DNA template. Additionally or alternatively, a universal capture oligonucleotide may be used that binds to a complementary adapter sequence added to DNA templates to allow a single type of capture oligonucleotide to be used to capture multiple DNA templates on a solid support. DNA templates may be arranged randomly or ordered in an array on a solid support, wherein each DNA template occupies a discrete position on the solid support.

Encoded polypeptide can be expressed and conjugated to a bead (e.g., via conjugation to the nucleic acid which is conjugated to the bead) by for example, starting with nucleic acid-coated beads (e.g., DNA-coated beads) prepared using the methods for displaying polynucleotides on beads. Conjugation of the polypeptide to the bead (e.g., directly or via attachment to the nucleic acid) may be performed in a microemulsion step. For example, DNA-coated beads are emulsified in a microemulsion, along with a mixture that includes reagents for cell-free in vitro transcription and translation (IVTT) methods resulting in the transcription and translation of the DNA on the beads and the production of the encoded polypeptide and/or protein. In some embodiments, the microemulsion contains reagents for IVTT as well as a catalytic enzyme or solution-phase DNA which codes for a catalytic enzyme and catalyzes the attachment of the polypeptide to the capture moiety on the nucleic acid. The components of the mixture can be tuned, as described herein, to ensure on average one DNA-coated bead and sufficient IVTT reagents.

In some embodiments, the nucleic acid in each droplet is amplified directly on the surface of the bead via extension of immobilized DNA oligos. In some embodiments, the nucleic acid may be separately amplified in a droplet containing no bead and then fused in a microfluidic channel with a separate droplet containing a bead. In some embodiments, upon generation of the emulsion droplets, the nucleic acid in each droplet is amplified via polymerase chain reaction to create a clonal population of each nucleic acid variant. Physical immobilization of the amplified nucleic acid in each microemulsion droplet can be achieved, e.g., via ligation or extension of immobilized DNA oligos to generate nucleic acid-coated beads (e.g., DNA-coated beads).

In one embodiment, the method further comprises amplification or extension of at least one DNA template. Amplification or extension may be performed using any known method, such as polymerase chain reaction (PCR) or other nucleic acid amplification process (e.g., ligase chain reaction (LGR), nucleic acid sequence-based amplification (NASBA), transcription-mediated amplification (TMA), Q-beta amplification, strand displacement amplification, or target mediated amplification). See, e.g., PCR Protocols, Vol. 226 (Methods in Molecular Biology, J. Bartlett and D. Stirling eds., Humana Press; 2nd edition, 2003; Wiedmann et al. (1994) PCR Methods Appl. 3(4):551-64; Deiman et al. (2002) Mol. Biotechnol. 20(2):163-179; Guatelli et al., Proc. Natl. Acad. Sci. USA (1990) 87:1874-1878 and J. Compton, Nature (1991) 350:91-92(1991); Hill (2001) Expert Rev. Mol. Diagn. 1:445-455; WO 89/1050; WO 88/10315; EPO Publication No. 408,295; EPO Application No. 8811394-8.9; WO91/02818; U.S. Pat. Nos. 5,399,491, 6,686,156, and 5,556,771; Walker et al., Clin. Chem. (1996) 42:9-13 and EPA 684,31; herein incorporated by reference in their entireties. In particular, clonal amplification methods such as, but not limited to bridge amplification, emulsion PCR (ePCR), or rolling circle amplification may be used to cluster amplified nucleic acids in a discrete area (see, e.g., U.S. Pat. Nos. 7,790,418; 5,641,658; 7,264,934; 7,323,305; 8,293,502; 6,287,824; and International Application WO 1998/044151 A1; Lizardi et al. (1998) Nature Genetics 19: 225-232; Leamon et al. (2003) Electrophoresis 24: 3769-3777; Dressman et al. (2003) Proc. Natl. Acad. Sci. USA 100: 8817-8822; Tawfik et al. (1998) Nature Biotechnol. 16: 652-656; Nakano et al. (2003) J. Biotechnol. 102: 117-124; herein incorporated by reference). For this purpose, DNA templates may include adapter sequences (e.g., adapters with sequences complementary to universal amplification primers or bridge PCR amplification primers) at the 5′ and 3′ends suitable for high-throughput amplification. For example, bridge PCR primers, attached to a solid support, can be used to capture DNA templates comprising adapter sequence complementary to the bridge PCR primers. The DNA templates can then be amplified, wherein the amplified products of each DNA template cluster in a discrete area on the solid support. In one embodiment, DNA templates are attached to a solid support, amplified, and sequenced prior to displaying ribosomal translation products for functional screening.

In various embodiments, microemulsion droplets may be used. Microemulsion droplets may be used to transform a bulk solution into multiple droplets. A droplet may contain reagents for reactions that may occur in the droplet and are separate from other microemulsion droplets or a bulk solution and allow for a microenvironment for a reaction to occur. For example, a conjugation, transcription, translation, or amplification reaction may occur in a microemulsion droplet. Methods for producing microemulsion droplets for the purpose of chemical and biochemical reactions are known to those of skill in the art. In general, microemulsion droplets contain an aqueous phase suspended in an oil phase (e.g. a water-in-oil emulsion). In an embodiment, the oil phase is comprised of 95% mineral oil, 4.5% Span-80, 0.45% Tween-80, and 0.05% Triton X-100. In some embodiments, the microemulsions are formed via direct mixing and/or vortexing of aqueous and oil phases. In some embodiments, the microemulsions are formed via a piezoelectric pump extruding the aqueous phase in a microfluidic channel containing oil phase. In some embodiments, the microemulsions are formed via mechanical mixing of aqueous and oil phases using a dispersing instrument or homogenizer. In an embodiment, each emulsion droplet contains on average a single primer-coated bead, one template DNA molecule, and a plurality of PCR primer molecules. Temperature cycling can be used to produce clonal DNA amplified from the template on the beads.

### Identification of Characteristics of Polypeptide Libraries

Polypeptide libraries may be generated and displayed as described elsewhere in this disclosure. The displayed polypeptides may be linked or otherwise associated with its corresponding polynucleotide from which the polypeptide is encoded by. Sequencing reactions may be performed on polynucleotides disclosed elsewhere herein. Any sequencing method may be used, including, but not limited to Maxam-Gilbert sequencing, Sanger sequencing (i.e., chain-termination method), sequencing-by-synthesis (SBS), sequencing-by-ligation, pyrosequencing, ion torrent sequencing, nanopore sequencing, and single-molecule real-time sequencing. In one embodiment, a plurality of DNA templates is sequenced by a high-throughput DNA sequencing method. See, e.g., Pettersson et al. (2009) Genomics 93 (2): 105-111; Maxam & Gilbert (1977) Proc. Natl. Acad. Sci. U.S.A. 74 (2): 560-564; Sanger et al. (1977) Proc. Natl. Acad. Sci. U.S.A. 74 (12): 5463-5467; Ronaghi et al. (1996) Analytical Biochemistry 242 (1): 84-89; Brenner et al. (2000) Nature Biotechnology 18 (6): 630-634; Schuster (2008) Nat. Methods 5 (1):16-18; Margulies et al. (2005) Nature 437: 376-380; Shendure et al. (2005) Science 309:1728-1732; Thompson et al. (2012) Electrophoresis 33(23):3429-3436; Merriman et al. (2012) Electrophoresis. 33(23):3397-3417; and Pareek et al. (2011) Journal of applied genetics 52 (4): 413-435).

The sequencing reactions may generate sequencing data for the polynucleotides. In some embodiments, the polynucleotides are attached to an array or solid support, or otherwise distinctly separated in space. By sequencing the polynucleotides, a particular polynucleotide on an array or solid support can be identified as having a particular sequence. As such a particular point on an array can be identified as having a particular or known sequence. Polypeptide display techniques as described in this disclosure allow for a polypeptide to be attached, linked, or otherwise associated with the polynucleotide that encodes the polypeptide. Since the sequencing reactions can identify a polynucleotide as having particular sequence, the amino acid sequence of a corresponding polypeptide can be determined.

Analysis of the polypeptides may be performed. Massively parallel high-throughput protein screening can be performed on the polypeptide libraries. For example, a multiplex assay can be performed where a library of polynucleotides can be immobilized on a solid support, such as on beads within confined locations of a carrier (e.g. capillary), or on the inner surface of a microchannel or flow chamber, or on the surface of a microscope slide, or the like. The surface can be a planar surface, or a coated surface. Additionally, the surface may comprise a plurality of microfeatures arranged in spatially discrete regions to produce a texture on the surface, wherein the textured surface provides an increase in surface area as compared to a non-textured surface.

Arrays may comprise a plurality or library of displayed ribosomal translation products, such as antigens, antibodies, enzymes, substrates, receptors, or regulatory molecules. Such arrays can be used, for example, in high throughput genetic or pharmacological screening, epitope mapping, protein engineering, or proteomic profiling. For high-throughput screening, arrays are preferably contained within a flow cell or a microfluidic device. Tens of millions to billions of proteins, peptides, or ribosomally translated small molecules potentially can be quantitatively screened simultaneously. Functional screening can be performed in a continuous flow or a stop-flow system, wherein the proteins are displayed on immobilized polynucleotides, as described herein, and different reagents and buffers are pumped into the system at one end and exit the system at the other end. Reagents and buffers may flow continuously or may be held in place for a certain period to allow ligand binding or enzymatic reactions to proceed. Additionally, ligands or substrates may be labeled to facilitate detection and quantitative analysis of binding interactions or enzymatic reactions.

In some embodiment, protein characterization assays are performed in a high-throughput sequencer. Ribosomal translation products (e.g., proteins or peptides, biologically active fragments thereof, or other ribosomally translated molecules) can be displayed on polynucleotides in a sequencer using the methods described herein, and then simultaneously characterized functionally directly on the sequencing flow cell. This may generate significant added value to high-throughput sequencing instrumentation, allowing high-throughput sequencing to readily be combined with protein screening.

In some embodiments, sequencing of the nucleic acid molecule and assaying the one or more functions or properties of each polypeptide are performed (e.g., sequentially, in any order) on the same machine, device, or instrument. In some embodiments, multiple assays are performed to determine two or more functions or properties of each polypeptide or multiple assays are performed to determine a single function or property of each polypeptide at varying condition. Multiple assays may be performed simultaneously or sequentially on the same machine, device, or instrument. For example, a single machine, device, or instrument may be used to sequence the nucleic acid molecule conjugated to each bead in order to identify the polypeptide conjugated to that bead; and to perform one or more assays to characterize each polypeptide (e.g., binding affinity, binding specificity, enzymatic activity, stability, e.g., at varying experimental conditions including, e.g., temperature and/or pH). In some embodiments, the sequencing and one or more assays produce fluorescence signatures that are measured by the single machine, device, or instrument.

The polypeptide characterization may comprise generating detectable signal based on the presence of a reaction or event. For example, a detectable signal may be generated upon the binding of a polypeptide to an antigen. The detectable signal may be a generated by a detectable label. The detectable label may be attached or coupled to an antigen (or target molecule) or may be attached to another reagent that can detect the antigen (or target molecule).For example, an antigen may be coupled to an enzyme that can generate a signal. The polypeptide library may be allowed to contact an antigen or target molecule and polypeptides may bind the antigen. After excess antigen is removed, the enzyme substrate is added and the enzyme may cause a detectable signal to be generated. The presence of the detectable signal may thereby indicate that a polypeptide has bound to the antigen, since the signal is generated when the enzyme attached to the polypeptide bound antigen is allowed to react with the enzyme substrate. Similarly, the antigen may be coupled to a fluorophore, and a signal may be generated upon excitation of the fluorophore. In another similar example, an antibody that binds to the antigen or target molecule may comprise an enzyme or fluorophore. The displayed polypeptide library may be allowed to interact with the antigen or target molecule. After removal of excess antigen, the antibody coupled to an enzyme or fluorophore is added and any excess is removed. Polypeptides bound to the antigen would be identifiable based on the generation of the signal, as the signal would be generated by the antibody bound to the antigen which was bound to the polypeptide.

The detectable label may be any composition detectable by spectroscopic, photochemical, biochemical, immunochemical, electrical, optical, or chemical means. Detectable labels may comprise fluorescent dyes (e.g., phycoerythrin, YPet, fluorescein, TagRFP, Texas red, rhodamine, green fluorescent protein, and the like, see, e.g., Molecular Probes, Eugene, Oreg., USA), quantum dots, radiolabels (e.g., 3H, 1251, 35 S, 14C, or 32P), enzymes (e.g., horseradish peroxidase, alkaline phosphatase and others commonly used in an ELISA), and colorimetric labels such as colloidal gold (e.g., gold particles in the 40-80 nm diameter size range scatter green light with high efficiency) or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; 4,366,241; 7,416,854; 8,114,681; 7,229,769; 6,846,645; 7,232,659; 6,872,578; 7,897,257; 6,730,521; 5,972,721; 7,498,177; 7,235,361; and 6,306,610; herein incorporated by reference.

Using the presence of a detectable signal, multiplexed quantitative protein assays may be performed. The multiplexed quantitative protein assays may allow for the calculation, generation or identification of a quantitative characteristic of the polypeptides. The quantitative characteristic may be a kinetic or thermodynamic parameter associated with the polypeptide. For example, the quantitative characteristic may be a measure of polypeptide stability, such as a melting (or denaturation) temperature (Tₘ) or a midpoint denaturation concentration (Cₘ), or an equilibrium constant. The quantitative characteristic may be a nonspecific binding potential, an aggregation potential, a hydrophobicity, a maturation time, or a protein expression level. The quantitative characteristic may be rate constant or kinetic parameter. The quantitative characteristic may be related to intramolecular or intermolecular interaction or reactions. For example, the quantitative characteristic may be a enzymatic reaction rate, enzymatic activity, fractional activity, or any associated thermodynamic constants. In some cases, multiplexed quantitative protein binding assays may be performed. The quantitative characteristic may be a binding affinity, association (Kₐ) or dissociation constant (K_{d}), a kinetic constant (e.g. a kₒₙ or k_{off} rate) of binding. A binding assay may be performed by observing detectable signals generated in the presence of binding event of a polypeptide of the library to a target molecule, and the intensity of the detectable signal may be used to quantify binding. By adding a series of known concentrations of target molecule, allowing binding of the target molecule to the polypeptide library and obtaining intensity data for each polypeptide, a binding curve can be generated for every polypeptide in the polypeptide library. This concentration dependent binding curve may be fit and a binding affinity for each polypeptide in the library can be calculated. For displayed polypeptides on an array, each polypeptide may be observed as a point on the array and the intensity of each point on the array at a given concentration of target molecule can be observed. In this way, multiple polypeptides may be analyzed in a same assay, and quantitative characteristics may be obtained for the multiple polypeptides in the assay.

The binding data or other data derived from the multiplexed quantitative protein assay can be used to characterize polypeptides in a polypeptide library. The polypeptide library may comprise variants of a reference or wild type sequence and these assays may characterize variants as having a neutral effect, a positive effect, or a negative effect on a characteristic of the polypeptide. For example, for characterizing a binding affinity, polypeptide variants may be characterized as having an increased binding affinity, decreased binding affinity, or minimally changed binding affinity to an antigen. For example, a neutral variation may have a dissociation constant greater than 0.25 times and less than 2 times a dissociation constant of a reference or starting polypeptide. A positive variation may have a dissociation constant less than or equal to 0.25 times a dissociation constant of a reference or starting polypeptide. A negative variation may have a dissociation constant greater than or equal to 2 times a dissociation constant of a starting or reference polypeptide. By using this data on quantitative characteristics, new polypeptide libraries can be constructed, for example, polypeptides that have combinations of multiple variants that had increased binding affinities. In addition, using quantitative measurements, the intensity or amplitude of the characteristics may be used to guide the construction of a future library, which data may be otherwise lost in a generic enrichment or selection assay. Additionally, the observation of variants that have negative or neutral effect may be actively observed, as opposed to being potentially lost in a generic selection or enrichment assay that only enriches for variants with a positive effect.

Multiplex quantitative protein assays as described herein may observe a large number of proteins in a given assay. The assays may observe the characteristics of 10³, 10⁴, 10⁵, 10⁶, 10⁷, 10⁸, 10⁹, or more polypeptides in a single assay or at a same time (or substantially the same time).The assays may be performed in a short amount of time. The assay may be performed in no more than 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 49 hours, 50 hours, 55 hours, 60 hours, 65 hours, 70 hours, or less.

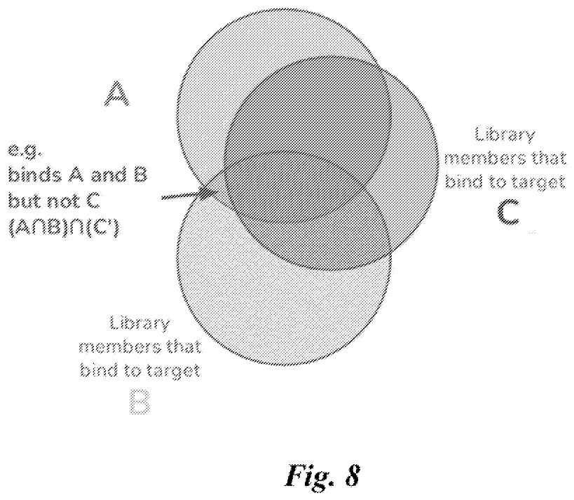

Multiple quantitative protein binding assays may be performed on a polypeptide library using different antigens or under different conditions. For example, a first binding assay may be performed using a first antigen to identify polypeptides that bind to the first antigen. A second binding assay may be performed using a second antigen to identify polypeptides that bind to the second antigen. Using the data generated from the two binding assay, polypeptide that bind to both the first antigen and the second antigen may be identified. The polypeptide library construction may be iterated as described elsewhere and synergistic combinations of variants may be identified as binding to both a first and a second antigen. Additionally, binding assay may be performed on a third antigen, a fourth antigen, or an nth antigen, and polypeptides that bind (or do not bind) to a particular set or subsets of antigens. Based on the data generated as well as iterative library design, polynucleotides that are specific to antigen(s) and do not bind (or have poor binding) to other antigens can be generated. For example, a polypeptide can be generated that binds a first and a second antigen and does not bind a third antigen. In another example, a polypeptide can be generated that binds a first and a second antigen and also binds a third antigen. FIG. 8 shows an example Venn diagram relating to the different types of polypeptides that may be generated relating to three antigens. A polypeptide may fall anywhere within this diagram such that it binds or doesn't bind (or has poor to minimal binding) with each of the antigens.

Identification of polypeptides that comprise a particular characteristic may be used to generate additional protein constructs or polypeptide conjugates. The polypeptides in a polypeptide library may represent functional domains or fragments of a full-length protein. Based on the sequences of the polypeptide (or corresponding polynucleotides), a polypeptide may be expressed that comprises the polypeptide that comprise a particular characteristic and a polypeptide sequence of another protein, domain, or fragment. For example, a polypeptide-chimeric antigen receptor fusion may be generated. A polypeptide drug conjugate (e.g. antibody drug conjugate) may be generated. For example, the polypeptides in the library may be heavy chain fragments, light chain fragments, nanobodies, or scFvs. Once a fragment has been identified as having a particular characteristic, a new full-length polypeptide comprising the sequence of the fragment may be generated. For example, full length antibody may be generated by expressing a polynucleotide comprising the encoding sequence of a Fc region along with encoding region of the fragment. For example, a CDR sequence may be identified based on the methods of the disclosure and a full-length IgG antibody may be generated based on the CDR sequence and sequences of a IgG backbone. For example, a bivalent nanobody may be generated based on the sequences of polypeptide analyzed by the methods in this disclosure. In this way, it may be possible to identify and generate full length antibodies (or other functional protein) based on data generated from the libraries that do not use full length proteins. This may be advantageous in that the construction of a protein of interest may be performed modularly and allow each domain of a protein to be individually characterized. For example, a library may be generated corresponding to a first CDR of antibody and methods of characterization may be performed on the library. A second library may be generated corresponding to a second CDR of antibody and methods of characterization may be performed on the second library. These libraries may be analyzed on a same sequencing chip or substrate or at a same time or different time. The CDR libraries may be subjected to different antigens or the same antigen, such that a multi-specific antibody, multi-epitopic, or highly specific antibody can be generated. Additionally, the smaller fragments may be easier to characterize or express on a given polypeptide display array.

Identification of polypeptides that comprise a particular characteristic may be used to generate additional polypeptide libraries. The polypeptides in a polypeptide library may represent functional domains with varying characteristics. For example, the polypeptides in a polypeptide library may comprise different binding affinities to an antigen. Based at least on the characteristics of a given polypeptide, additional libraries may be generated to optimize or improve a characteristic. For example, a polypeptide in the library may show a moderate or low affinity to an antigen. A subsequent library may use the polypeptide with a moderate affinity and generate a plurality of polypeptides comprising point mutants of the polypeptide or fusions comprising the polypeptide. Because the original polypeptide demonstrated a moderate to low affinity, point mutants or fusions that improve on the affinity may be more easily identifiable, as compared to using an original polypeptide that already had high affinity to an antigen. Data obtained regarding constructs with improved affinity (or other characteristics) may be used to generate further improved construct. For example, a fusion protein comprising a first domain with moderate binding and a second domain with moderate binding may demonstrate an avidity effect. The first domain may be “swapped” to a domain with higher affinity to generate a polypeptide construct with increased binding, avidity, or a combination of both. Libraries may also comprises fusion polypeptides or constructs that have a domain that does not bind or has low affinity to bind to an antigen. For example, a fusion polypeptide may have a first domain that binds and a second domain that does not bind. The presence of the domain, or monomer, that does not bind may allow for a polypeptides characteristic to be compared against another polypeptide with more similar physical characteristics. In the example of a polypeptide that has a first domain that binds and a second domain that does not bind, this may be directly compared to a polypeptide with same first domain but with a second domain that does bind. These polypeptides may be of a more similar size, length, shape as compared to a polypeptide that only has one domain. As such, the comparison may lead to more accurate result. The domain or polypeptide region that does not bind (or has minimal or no affinity to an antigen) may have a length, size, shape, net charge that is the same as a domain that does bind or have affinity to an antigen. The domain or polypeptide region that does not bind (or has minimal or no affinity to an antigen) may have a length, size, shape, net charge that is substantially same as a domain that does bind or have affinity to an antigen. The domain or polypeptide region that does not bind (or has minimal or no affinity to an antigen) may have a length, size, shape, net charge that no more than 10% different than a domain that does bind or have affinity to an antigen.

Polypeptides generated from the methods of the present disclosure may use quantitative characteristics analyzed in different libraries to generate optimized polypeptides. For example, first library may generate data relating to binding affinity for a plurality of point mutation of a first scaffold. A second library may generate data relating to binding affinity of plurality of different scaffolds including the first scaffold. A third library may comprise data relating to binding affinity from combinations of any two scaffolds of the second library. A polypeptide may be generated that comprises two scaffolds with point mutations that were analyzed in the first library. In this way an optimized polypeptide may be generated that leverage information gathered at a first level of detail (e.g., point mutations for a given scaffold) and information gathered at a second level of detail (e.g., bi-valent or bi-epitopic scaffolds) to generate a polypeptide which was not necessarily present in its entirety in a given library.

For example, a first library may comprise a plurality of single domains that bind to an antigen. A second library may comprise point mutations of one or more single domains of the plurality of single domains in the first library. The first library may allow identification of a first scaffold that binds to an antigen. The second library may generate variants of the first scaffold that have different binding characteristics. Determining the binding characteristics (or other quantitative characteristic) may be used to generate a new library, or a separate library may also be assayed simultaneously without using data generated from a prior generated library. The generated second library may identify mutations that generate a desired or target binding characteristic. For example, the binding characteristic may be an improvement on the binding. A third library may be generated which combines the single domains into fusion polypeptides comprising pairs of single domains. The third library may comprise all possible combinations of single domain pairs. The third library may comprise all possible permutations of single domain pairs. The third library may comprise single domain pairs wherein a single domain has a reduced binding characteristic as compared to a reference or wild-type single domain. The third library may be used to identify bi-epitopic binder and the use of single domains with reduce binding may allow the bi-epitopic binder to be more easily identified. As the bi-epitopic binder may significantly increase the binding characteristics based on avidity effects, the use of two strong binder in the construct may cause the increase in binding to be difficult to resolve or identify. By using a weaker binder that still binds to an epitope, the avidity effects gained in the bi-epitopic construct may be more readily apparent and may be assayable using a given binding assay. The information generated by each library may be combined to generate an optimized polypeptide, wherein the optimized polypeptide was not necessarily analyzed in any of the libraries. For example, the library comprising constructs with two or more domains may be used to determine and identify domains or scaffolds that bind in tandem or a bi-epitopic. The data obtained using a library comprising point mutations of scaffolds may identify mutation that cause a high or highest binding affinity to an antigen. The mutation may then be substituted in to the bi-epitopic construct to generate a bi-epitopic (or multi-epitopic) construct where each domain has an optimized binding affinity or binding characteristic.

Fragments analyzed using the methods of the present disclosure may be used to generate larger polypeptides, such as fusion proteins. Libraries may be generated to encode and generate the larger polypeptides. For example, a library may be generated that encodes fusion proteins. The larger polypeptides may be generated without generating a library. For example, data pertaining to a scFv or CDR may be generated using the methods and systems disclosed elsewhere herein, and a full length antibody may be generated using this data without the use of a library encoding for a full length antibody.

The polypeptides may comprise a linker or spacer domain. The linker may link two domains to form a fusion protein. The linker may be a polypeptide linker. The linker or spacer domain may comprise at least 2, 3, 4, 5, 6, 7, 8, 9, 10, 11, 13, 14, 15, 16, 17, 18, 19, 20, 21, 22, 23, 24, 25, 26, 27, 28, 29, 30, 31, 32, 33, 34, 35, 40, 45, 50, 60,70, 80,90, 100, or more amino acids. The linker or spacer domain may comprise no more than 2, 3, 4, 5, 6, 7, 8, 9, 10, 11, 13, 14, 15, 16, 17, 18, 19, 20, 21, 22, 23, 24, 25, 26, 27, 28, 29, 30, 31, 32,33, 34, 35, 40, 45, 50, 60,70, 80,90, 100, or less amino acids. The spacer domain may be a polypeptide spacer domain. The spacer domain may be a N-terminal spacer domain. The spacer domain may be a C-terminal spacer domain. A spacer domain or linker may comprise a positive, negative, or neutral charge. A spacer domain or linker may comprise a net positive, net negative, or net neutral charge. A spacer domain or linker may be hydrophobic, hydrophilic, or partially hydrophobic or hydrophilic. For example, a first VHH may be analyzed using methods described and libraries corresponding to the first VHH (e.g. libraries of single point mutations). Once analysis of the first VHH is performed, certain VHHs comprising particular characteristics (such as binding to a target or epitope) may be used to generate a second library comprising a combination of another VHH separated by a linker sequence. The other VHH may be analyzed by creating a library, such that both VHHs are independently analyzed and selected for, prior to generation of a subsequent library comprising constructs comprising multiple VHHs. The library comprising constructs comprising two or more VHHs separated by a linker sequence(s) may then be subjected to analysis as described elsewhere herein. In this way bi-epitopic constructs may be generated, where each binding unit is individually, or simultaneously analyzed to identify a construct with desirable parameters or certain characteristics. The libraries may also be analyzed or generated independently and may be assayed simultaneous or sequentially. For example, a library comprising constructs of two of more VHHs may be generated and tested along with a library comprising constructs of single VHHs, without data from the single VHH library guiding, or being used to dictate the polypeptides of the library comprising constructs of two or more VHHs.

The libraries may comprise generating of polypeptides that have different linker or spacer domains. A library may comprise polypeptides comprising a scaffold or domain and a N-terminal spacer, wherein the polypeptides have different N-terminal spacers. The N-terminal spacer may alter the display or other characteristic of the polypeptides, and the library of different N-terminal spacers may allow for the determination of an optimal or preferred N-terminal spacer for a given polypeptide or scaffold. Similarly, libraries may be generated and assayed for N-terminal spacers, C-terminal spacers, linkers, or a combination thereof. The N-terminal spacers, C-terminal spacers, or linkers may comprise differing lengths, charges, flexibility, steric bulk, hydrophobicity, or other characteristic that may affect the characteristic of the polypeptide. The libraries may allow for the selection of appropriate spacers and linker for a polypeptide construct. In the context of bi-epitopic (or multi-epitopic) binders, varying length of linkers may affect the binding properties. As epitopes for an antigen may be a specific distance apart, the spatial characteristics of binders may be relevant for optimizing bindings. For example, a linker separating two binding domains that is too short may cause the binder to be unable to engage both binding domains on an antigen at the same time, thereby affecting the overall binding capability. As such, libraries containing a same two scaffolds or binding domains with different linkers may be used to identify an optimal or appropriate linker.

In various aspects, data is generated or obtained that may be used to generate a polypeptide. For example, data pertaining to the binding characteristics of a plurality of polypeptides may be generated or obtained. This data may be used to guide the design of a library. For example, a first library of different scaffold may be generated and data pertaining to the binding characteristics of the scaffolds may be generated. The scaffolds that did not bind to an antigen may be omitted from future libraries. Scaffolds that bind the antigen may be used a reference scaffold or polypeptide for generating a library of point mutants of that scaffold. The data may be obtained from publicly available databases. For example, publicly available data on polypeptide that binds to an antigen may be used to determine a reference polypeptide or scaffold. Multiple data sets may be used and compared. For example, data pertaining to polypeptides comprising a single domain may be compared with data pertaining to polypeptides comprising fusions of single domains. By comparing the data of the single domain to a corresponding polypeptide comprising the same single domain, improvements to the binding based on the addition of another domain (e.g., bi-epitopic constructs) may be determined.

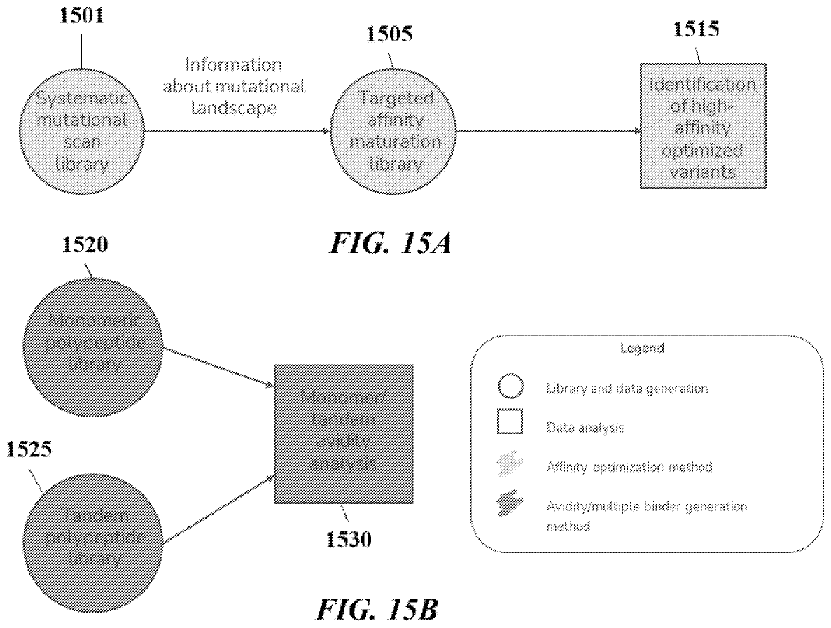

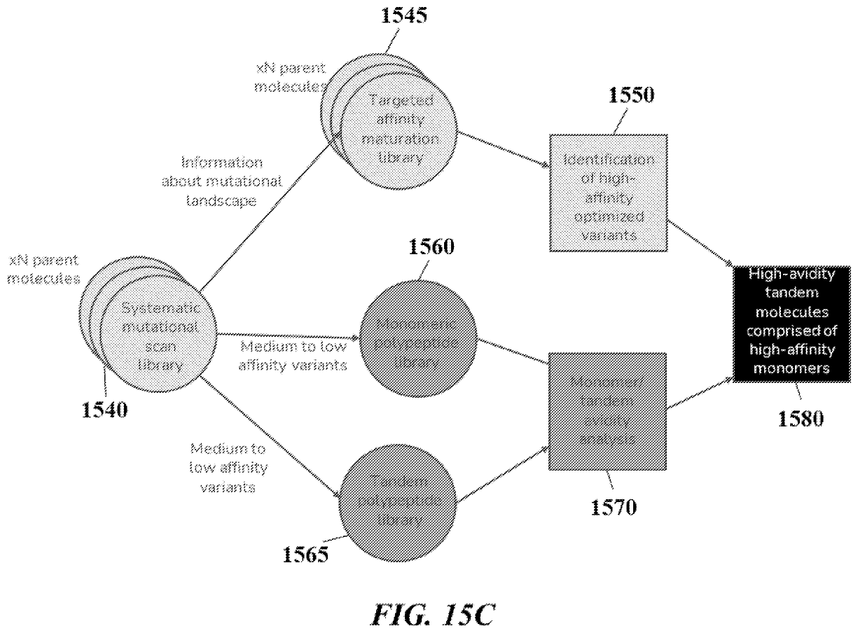

FIG. 15A-15C show example schematic workflows that may be used to generate libraries and use data derived from libraries to generate a polypeptide of interest. FIG. 15A shows a schematic workflow that allows for the generate of affinity optimized variants. An initial library 1501 is generated which comprises mutations of a polypeptide. The library may be an systemic mutational scan library in which a single point mutation substituting each of all 20 canonical amino acids is made at every residue from an area of the polypeptide. Analysis of library 1501 generates information about the mutational landscape of a polypeptide where the effect of an individual mutation can be analyzed. Using the analysis of the data, a 2nd library 1505 is generated that has “targeted” based on information discovered in library 1501. For example, library 1505 may comprise mutations to multiple residues identified in library 1501 that could lead to improved binding is generated. The initial library 1501 may for example identify single point mutations that increase binding affinity. Library 1505 may comprise polypeptides with multiple single point mutations that were identified in in library 1501. The initial library 1501 may for example identify residues which are amenable to mutations in which, for example, some or all single point mutations result in a neutral or positive increase in binding. The library 1505 may have polypeptides with every combination of mutations at residues identified as potentially amenable to mutation. The screening of library 1505 may allow for the generation of large data set of different polypeptide that are multiple mutations away from the initial reference or wild-type polypeptide. Data analysis 1515 is performed on this data set may allow the identification of the affinity optimized variant.

FIG. 15B shows an example schematic to identify tandem pairs that lead to increase of avidity. A first library 1520 of monomeric polypeptides that can bind to an antigen is generated and data for different individual monomeric polypeptides is generated. A second library 1525 is also generated that comprises polypeptides that are made by creating fusion tandem polypeptides comprising the polypeptide sequences of two monomeric polypeptides. The second library 1525 may have every possible permutation of two monomeric polypeptides. The libraries 1520 and 1525 may also comprise polypeptides with different N-terminal spacers and/or C-terminal spacer which may affect the binding and display of the polypeptide. Additionally, second library 1525 may also comprise different linkers between the two monomeric polypeptides. For example, the second library 1525 may comprise a polypeptide with two monomeric polypeptides with a linker, and a second polypeptide with the same two monomeric polypeptides with a different linker. Additionally, the library 1525 may comprise polypeptides that have one monomeric polypeptide that can bind to the antigen and another monomer that does not bind to the antigen. This may generate a polypeptide that acts as a baseline to compare against other tandem polypeptides as it is a similar size but only has one binding domain, creating a “pseudo-monomer”. Data analysis 1530 is performed by comparing the data from monomeric polypeptide library 1520 and data from the tandem library 1525 (and pseudo-monomers) to find pairs in the tandem library that resulted in an increase in binding affinity as compared to its component individual monomers (and pseudo-monomers).

FIG. 15C shows a schematic of an example workflow that combines the analysis and libraries described and illustrated in FIGS. 15A and 15B. A set of libraries and data 1540 is generated for multiple reference or wild-type molecules. For each of these polypeptides an initial systemic mutational scan library, such as library 1501, is generated. Analysis of libraries 1540 generates information about the mutational landscape of a polypeptide where the effect of an individual mutation can be analyzed. Information about the mutational landscape can then be used to generate 3 different libraries. Similar to as described for library 1505, targeted libraries are generated for each reference or wildtype polypeptide. Using the analysis of the data, another set of libraries 1545 is generated that has “targeted” based on information discovered in library 1540. For example, library 1545 may comprise mutations to multiple residues identified in library 1540 that could lead to improved binding is generated. The set of libraries 1540 may for example identify single point mutations that increase binding affinity. Libraries 1545 may comprise polypeptides with multiple single point mutations that were identified in in libraries 1540. The libraries 1540 may for example identify residues which are amenable to mutations in which, for example, some or all single point mutations result in a neutral or positive increase in binding. The libraries 1545 may have polypeptides with every combination of mutations at residues identified as potentially amenable to mutation. The screening of libraries 1545 may allow for the generation of large data set of different polypeptide that are multiple mutations away from the initial reference or wild-type polypeptide. Data analysis 1550 is performed on this data set may allow the identification of the affinity optimized variants. A second library 1560 is generated that comprises multiple monomers that demonstrated medium to low affinities, as determined by sets of libraries 1540. A third library 1565 is also generated that comprises polypeptides that are made by creating fusion tandem polypeptides comprising the polypeptide sequences of two monomeric polypeptides. The second library 1565 may have every possible permutation of two monomeric polypeptides. The libraries 1560 and 1565 may also comprise polypeptides with different N-terminal spacers and/or C-terminal spacer which may affect the binding and display of the polypeptide. Additionally, second library 1565 may also comprise different linkers between the two monomeric polypeptides. For example, the second library 1565 may comprise a polypeptide with two monomeric polypeptides with a linker, and a second polypeptide with the same two monomeric polypeptides with a different linker. Additionally, the library 1565 may comprise polypeptides that have one monomeric polypeptide that can bind to the antigen and another monomer that does not bind to the antigen. This may generate a polypeptide that acts as a baseline to compare against other tandem polypeptides as it is a similar size but only has one binding domain, creating a “pseudo-monomer”. Data analysis 1570 is performed by comparing the data from monomeric polypeptide library 1560 and data from the tandem library 1565 (and pseudo-monomers) to find pairs in the tandem library that resulted in an increase in binding affinity as compared to its component individual monomers (and pseudo-monomer). Data analysis 1580 is then performed to identify a high affinity tandem binder based on data analysis 1550 and data analysis 1570. Data analysis 1570 has identified the monomers that bind in tandem, however each monomer itself as generated may not have a high affinity. Data analysis 1550 has determined the mutations that lead to an increase affinity in a given monomer construct. By combining the data, and adding in the mutations into each of the monomers of the tandem pair discovered in data analysis 1570, a tandem binder where each monomer has high affinity can be generated.

As multiplex protein assays may be performed and imaged on a protein array, fiducial markers may be used. Fiducial markers may allow for the alignment of a plurality of images from a given array. As the multiplexed protein assays comprises many polypeptides on a given array, it may be advantageous to prevent a polypeptide from being mistaken for another polypeptide. By imaging one or more fiducial markers along with the polypeptides, a position on the array may be identified as the location of a fiducial marker. The signals for the polypeptides on the array may be reference against the one or more fiducial markers, thereby allowing the location of each polypeptide to be mapped accurately. For a binding assay, multiple images of a polypeptide array may be generated. These images may be aligned based on the position of the one or more fiducial markers.

Fiducial markers may be generated by capturing a fiducial polynucleotide on the array. A polynucleotide complementary to the fiducial polynucleotide may then be added, where the polynucleotide complementary to the fiducial polynucleotide comprises a detectable label. This detectable label may act as a fiducial marker.

In various embodiments, the polypeptides libraries are allowed to bind to antigens and binding data is derived for the polypeptide libraries. An antigen may be a small molecules, a protein or polypeptide, a receptor, a hormone, or any molecule. The antigen may be derived from an animal, plant, fungi, microbe, virus, or other biological organism. The antigen may be an inorganic compound or organic compound. The antigen may be derived or generated from a pathogen. For example, the antigen may be derived or generated by SARS-CoV-2. The antigen may be SARS-CoV-2 receptor binding domain (RBD).

The polypeptides generated using the methods, compositions, and system described in this disclosure may be used for generating antibodies or antibody fragments. Antibodies and antibody fragments may be used as therapeutics or diagnostics, and antibodies with high affinities and/or high specificity may be highly useful. The methods, compositions, and systems provided elsewhere herein may be able to generate antibodies with high affinity and/or high specificity. Additionally, due to the multiplexing capabilities of the methods described, antibodies of particular characteristics may be assayed and designed in a highly efficient manner.

### Computer Control Systems

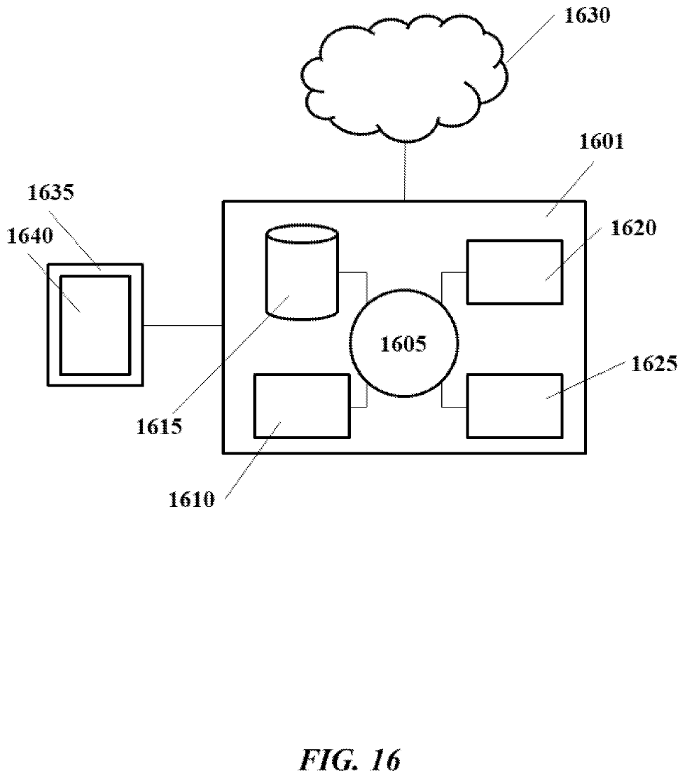

The present disclosure provides computer control systems that are programmed to implement methods of the disclosure. FIG. 16 shows a computer system 1601 that is programmed or otherwise configured to perform parts of the methods, such as process images, or calculate binding affinities corresponding to the polypeptide libraries. The computer system 1601 can regulate various aspects of the methods of the present disclosure, such as, for example, receive images, process images for intensities, output binding curve. The computer system 1601 can be an electronic device of a user or a computer system that is remotely located with respect to the electronic device. The electronic device can be a mobile electronic device.

The computer system 1601 includes a central processing unit (CPU, also “processor” and “computer processor” herein) 1605, which can be a single core or multi core processor, or a plurality of processors for parallel processing. The computer system 1601 also includes memory or memory location 1610 (e.g., random-access memory, read-only memory, flash memory), electronic storage unit 1615 (e.g., hard disk), communication interface 1620 (e.g., network adapter) for communicating with one or more other systems, and peripheral devices 1625, such as cache, other memory, data storage and/or electronic display adapters. The memory 1610, storage unit 1615, interface 1620 and peripheral devices 1625 are in communication with the CPU 1605 through a communication bus (solid lines), such as a motherboard. The storage unit 1615 can be a data storage unit (or data repository) for storing data. The computer system 1601 can be operatively coupled to a computer network (“network”) 1630 with the aid of the communication interface 1620. The network 1630 can be the Internet, an internet and/or extranet, or an intranet and/or extranet that is in communication with the Internet. The network 1630 in some cases is a telecommunication and/or data network. The network 1630 can include one or more computer servers, which can enable distributed computing, such as cloud computing. The network 1630, in some cases with the aid of the computer system 1601, can implement a peer-to-peer network, which may enable devices coupled to the computer system 1601 to behave as a client or a server.

The CPU 1605 can execute a sequence of machine-readable instructions, which can be embodied in a program or software. The instructions may be stored in a memory location, such as the memory 1610. The instructions can be directed to the CPU 1605, which can subsequently program or otherwise configure the CPU 1605 to implement methods of the present disclosure. Examples of operations performed by the CPU 1605 can include fetch, decode, execute, and writeback.

The CPU 1605 can be part of a circuit, such as an integrated circuit. One or more other components of the system 1601 can be included in the circuit. In some cases, the circuit is an application specific integrated circuit (ASIC).

The storage unit 1615 can store files, such as drivers, libraries and saved programs. The storage unit 1615 can store user data, e.g., user preferences and user programs. The computer system 1601 in some cases can include one or more additional data storage units that are external to the computer system 1601, such as located on a remote server that is in communication with the computer system 1601 through an intranet or the Internet.

The computer system 1601 can communicate with one or more remote computer systems through the network 1630.For instance, the computer system 1601 can communicate with a remote computer system of a user.Examples of remote computer systems include personal computers (e.g., portable PC), slate or tablet PC's (e.g., Apple®iPad, Samsung® Galaxy Tab), telephones, Smart phones (e.g., Apple®iPhone, Android-enabled device, Blackberry®), or personal digital assistants. The user can access the computer system 1601 via the network 1630.

Methods as described herein can be implemented by way of machine (e.g., computer processor) executable code stored on an electronic storage location of the computer system 1601, such as, for example, on the memory 1610 or electronic storage unit 1615. The machine executable or machine readable code can be provided in the form of software. During use, the code can be executed by the processor 1605.In some cases, the code can be retrieved from the storage unit 1615 and stored on the memory 1610 for ready access by the processor 1605.In some situations, the electronic storage unit 1615 can be precluded, and machine-executable instructions are stored on memory 1610.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1601, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. On-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1601 can include or be in communication with an electronic display 1635 that comprises a user interface (UI) 1640 for providing, for example, providing the sequences of polypeptides, or the concentration of antigens for each image. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1605. The algorithm can, for example, generate sequences of polypeptides, calculate binding coefficient, or fit curves.

### EXAMPLES

### Example 1: Generation of Nanobodies

Nanobodies (or VHHs) are a class of single domain antibodies found in camelid species including camels, llamas and alpacas. Comprised of a single variable heavy chain, nanobodies exhibit high specificity and affinity to their antigenic targets, and often have favorable immunogenicity and toxicity profiles. Due to their small size (˜15 kDa), they are easier to produce and potentially more stable than conventional antibodies. These properties have made nanobodies an exciting target for developing novel therapeutics. Indeed, since their discovery in the 1990s, nanobodies are increasingly entering clinical trials as drug candidates to combat various diseases including numerous cancers, thrombotic thrombocytopenic purpura, inflammation, and Alzheimer's, among others.

Since late 2019, nearly 2 million people have died in a global pandemic caused by SARS-CoV-novel coronavirus that has infected more than 80 million people around the world. The viral envelope is studded with numerous copies of a spike protein that binds the angiotensin-converting enzyme 2 (ACE2) receptor on human epithelial cells, thereby initiating viral entry. A number of groups have focused, therefore, on developing affinity reagents capable of binding this spike protein, and several VHH sequences have been reported that exhibit both high-affinity binding to the spike protein, and high levels of neutralization of viral entry in vitro. Furthermore, pharmaceutical companies have already started trials to test the efficacy of spike-binding nanobodies.

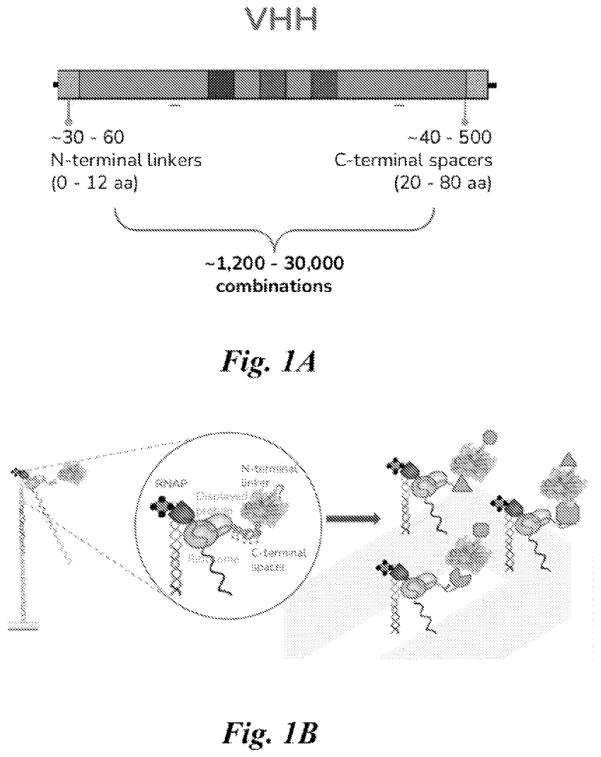

Sy62 is an anti-SARS-CoV-2 VHH, previously described in the literature. Sy62 has a high signal-to-noise and superb binding affinity (apparent K_{D} of ˜3.4 nM) and was used as a reference sequence for generating variants. Initial optimization of display was performed by generating polypeptide libraries with different spacer and a linker regions. A variety of C-terminal spacers and n-terminal linkers were screened. Screening of successful display is analyzed by observing the proper folding and function of the VHH on the display chip. FIG. 1A shows a schematic for display screening, where˜-1,200-˜30,000 combinations are displayed and analyzed for binding. FIG. 1B shows an example schematic of polypeptides of the library displayed using ribosomal display, wherein different shapes are representative of different N-terminal linkers and C-terminal spacers that can be displayed.

Individual amino acid contributions to binding within the complementary-determining regions (CDR) regions of Sy62 were then analyzed by making large, targeted mutational libraries, and then measuring the effects of each mutation on binding, as well as characterizing cooperative interactions between mutations.

Such analysis yields comprehensive catalogs of functional mutations within the Sy62 CDRs and provides a handle for affinity modulation and improvement. To generate these data sets, a multi-pronged approach was used. In a first experiment, the mutant affinity landscape of Sy62 CDRs with ˜90,000 distinct variants divided into 3 distinct sub-libraries. The first sub-library included an exhaustive set of single mutants in which each CDR residue was mutated to all possible 20 amino acids using degenerate NNK codons. In the second sub-library, compensating mutations between interacting residues in Sy62 CDRs were identified. By analyzing the crystal structure of a parental nanobody from which Sy62 was derived, candidate intra- and inter-CDR-interacting residues were identified and then pairs of residues were mutated to all possible double-mutant combinations. The third and final sub-library explored the dependence of Sy62 binding affinity on the length of CDR3 with single residue insertions at each position in addition to all possible deletions ranging in length from 1-17 amino acids. These three CDR sub-libraries were each embedded into 6 different framework scaffolds that consisted of the wild-type (WT) Sy62 frameworks (FRs) with some diversity introduced in 4 critical residues in the FR2 framework region. The libraries were constructed by generating a plurality of polynucleotides encoding for the polypeptide variants and then using ribosome display on a sequencing chip.

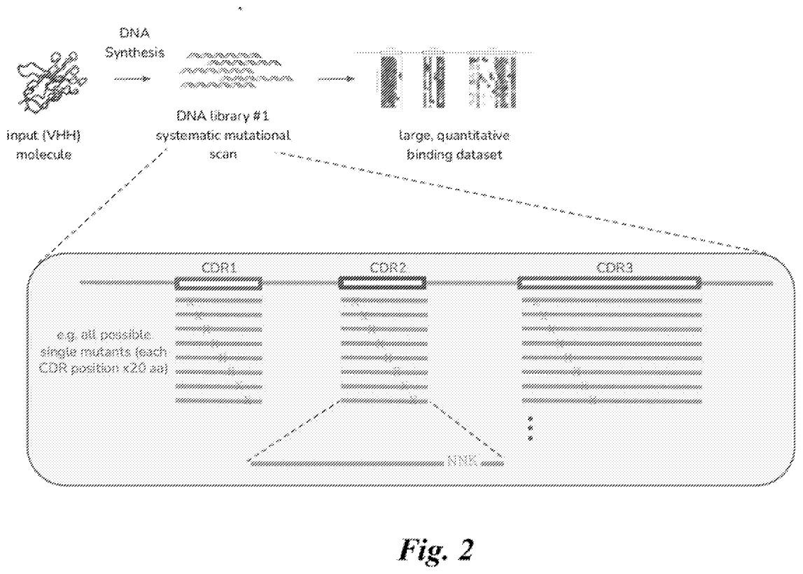

FIG. 2 shows a schematic for the general workflow relating to the first sub-library, in which a DNA library in generated for every single point mutation and then quantitative analysis can be performed. Specifically, analysis of the first sub-library was performed by displaying the polypeptides of a sub-libraries on a sequencing chip. Initially, a library of polynucleotides encoding for the polypeptides was added and captured onto a sequencing chip. The polynucleotides were sequenced to determine the location of the chip of each polynucleotide and subsequently displayed corresponding polypeptide. Reagents for ribosomal display were added (e.g. RNA polymerase, dNTPs, ribosomes, tRNAs) such to display a corresponding VHH polypeptide from each polynucleotide. To analyze binding, different concentrations of labeled SARS-CoV-2 RBD were added to the sequencing chip and allowed to bind to the displayed VHH polypeptides and excess SARS-CoV-2 RBD was removed. Fluorescent signal from the labeled SARS-CoV-2 RBD was generated and the intensity of each polypeptide was collected by imaging of the sequencing chip. By generating an image of the chip for each concentration of labeled SARS-CoV-2 RBD, a binding curve for each polypeptide on the chip was generated. The binding curve can then be fitted to determine a binding coefficient or other quantitative binding measure.

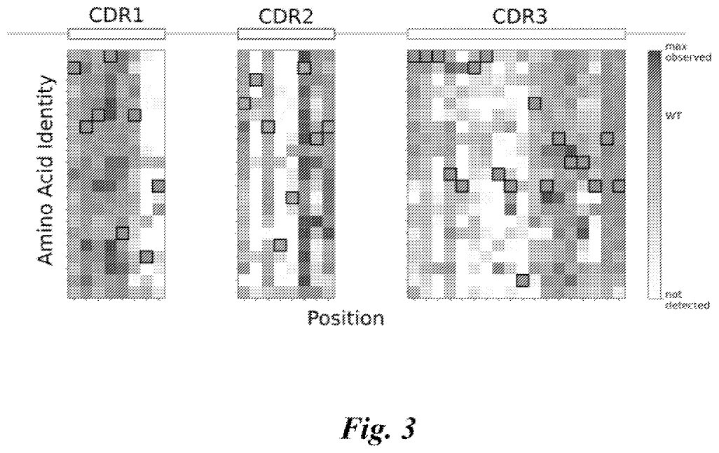

Protein display on a massively parallel array (Prot-MaP) analysis of the first sub-library revealed strong binding signals and diverse binding constants as well as a complex dependency of the CDRs on both amino acid position and identity. Certain residues were observed to be mutagenized without effects on binding, whereas other residues only allowed mutations to specific other amino acids. Furthermore, some amino acids that increase binding when mutated. Indeed, residue CDR2.6 showed improved activity when mutated away from WT to any of ˜15 different amino acids. Further, the second sub-library validated a structure-guided approach by not only affirming that target-interacting residues are highly sensitive to mutation but allowing us to identify compensatory mutations that restored function in otherwise-dead single-mutants, providing a potential way of optimizing even highly-sensitive residues. FIG. 3 shows a heat map of the binding data colored by apparent Kd (K_{d}^{app}) Specifically, single mutant CDR variants for each VHH were first grouped and binned by the sequence of their specific parent CDRs. The binding data for each set of CDR mutants was then organized as individual heatmaps with the residues constituting the CDR arrayed on the x-axis and the identities of the 20 individual amino acids (that each position was mutated to) on the y-axis. The WT amino acid identities at each position were marked by black boxes on the heatmap. Binding affinities of the variants in the heatmap are colored from light (weak affinity) to dark red (high affinity). Variants for which no binding was observed even at the highest tested concentration are shown in white while the highest affinity variants are colored purple. The variants could be grouped as neutral (Kd=1.5-7 nM), negative (Kd>7 nM), or positive (Kd<=1.5 nM), based on the wild type Kd of 3.4 nM

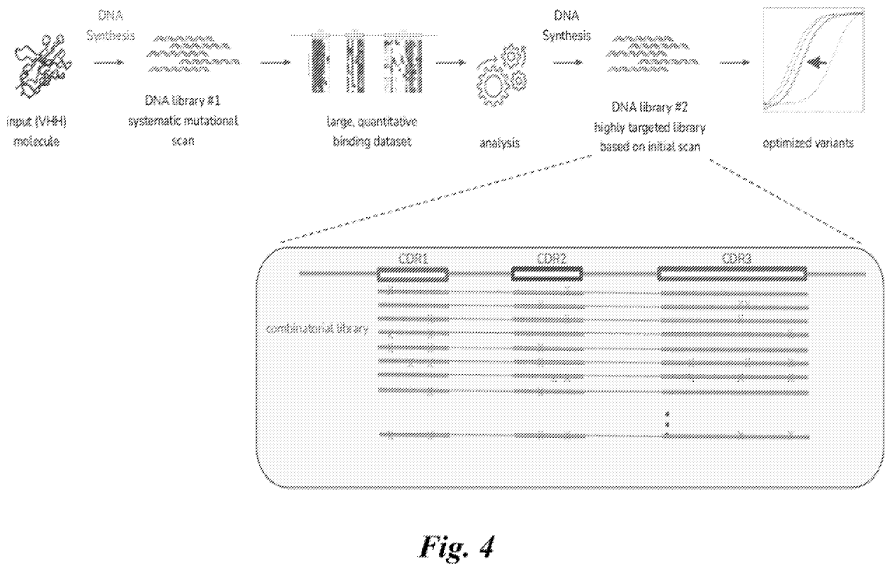

In the second step of the process, having found variants of Sy62 capable of maintaining high affinity binding across a diverse mutational landscape via single mutant analysis, 21 mutations at 13 positions out of the 34 total residues in the CDRs were selected that showed equal or improved signal and binding affinity compared to the wild-type. This second library explored all possible combinations of anywhere from 1 to all 13 positions simultaneously mutated to all possible combinations of these neutral-to-beneficial (when considered individually) mutations at the amenable, resulting in a library comprising ˜200,000 Sy62 variants. FIG. 4 shows a corresponding schematic for the generic workflow, in which a first DNA library in generated and then quantitative analysis is performed. Using the data from the first DNA library a second DNA library can be generated and quantitative analysis can be performed to generate an optimized variant.

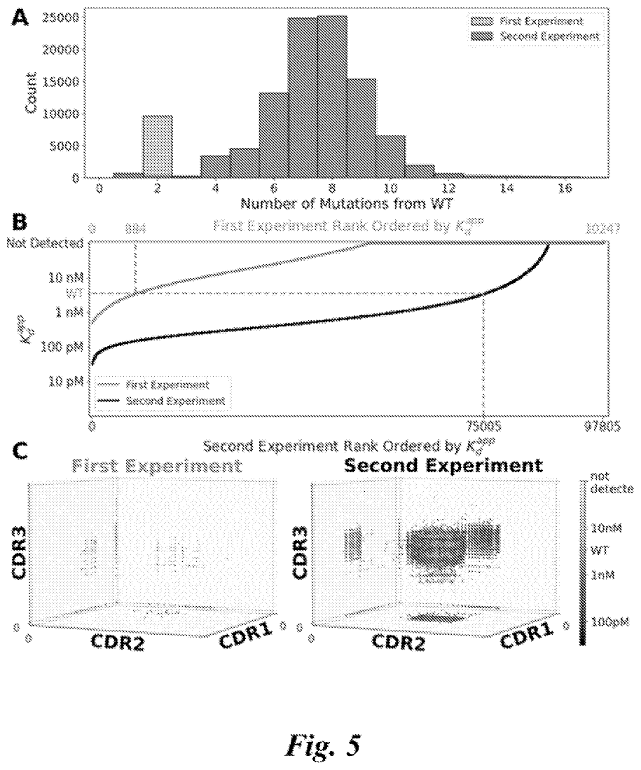

Upon sequencing and Prot-MaP analysis of the library comprising ˜200,000 Sy62 variants, variants that were surprisingly distant in sequence space—13 mutations away from wild-type (WT)—were identified and performed equal to or better than their parental sequence. FIG. 5 shows the results from analysis of the initial sub-libraries (“first experiment”) and the results from the library generated based on the variants identified in the initial sub-libraries (“second experiment”). FIG. 5A shows Sy62 CDR variants from each of the two experiments were plotted as a frequency histogram binned by the number of mutations observed in each experiment. In the first experiment (blue bars), most variants were one to three mutations away from the WT sequence. The neutral and beneficial mutations from this library were then combined in a second experiment (black bars) in multiple different permutations to generate a diverse combinatorial library of variants that were between 3-17 mutations away from the WT sequence. Most members of the second library contained between 6-8 mutations away from WT. FIG. 5B shows the apparent binding affinities (y-axis) of variants from each of the two experiments (first experiment denoted by blue lines; second experiment denoted by black lines) were ranked from highest to lowest affinity and plotted as a function of the ranks (x-axis). In each experiment, the rank of the WT sequence is marked by red dashed lines. In the first experiment, less than 9% of the variants had affinities that were improved over WT. The affinity maturation process resulted in a nearly 9-fold increase (˜8.7% to ˜77%) in the number of variants with greater affinity to ligand than WT between the two experiments. FIG. 5C shows the apparent binding affinities of Sy62 variants from the first (left panel, blue) and second (right panel, black) experiments were plotted individually on 3-dimensional scatter plots as a function of the mutation distance of each CDR from the Sy62 WT sequence. Apparent binding affinities of the variants are colored from light (weak affinity) to dark (high affinity).

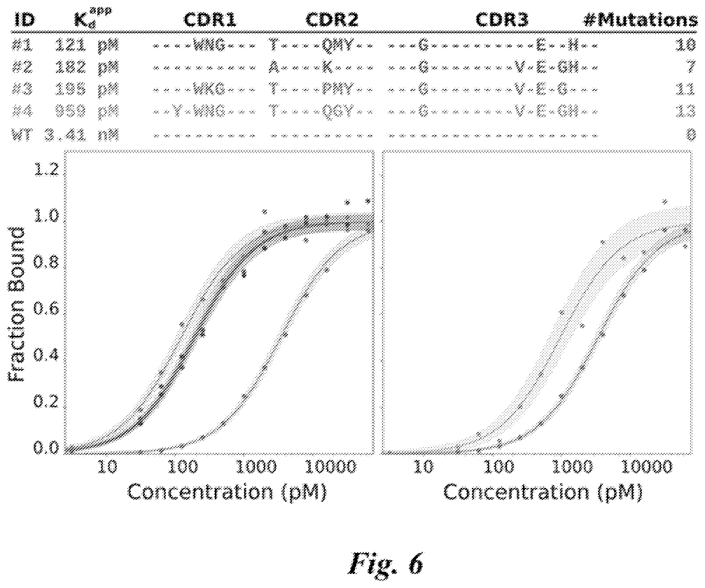

Some of the highest-affinity variants identified were 7-11 mutations away from WT. FIG. 4 shows select high affinity (arrow) and highly-mutated (grey) variants that outperformed the WT Sy62 nanobody (black).Fluorescence binding data of variants from the combinatorial library (second experiment) were fit to a 1:1 equilibrium binding model. FIG. 6 shows the ligand bound (y-axis) as a function of ligand concentration (x-axis) with shaded regions indicating ±standard deviation in each fit parameter. The left panel shows select variants (left curve) with 17-28 fold higher binding affinities than WT Sy62 (right curve). These variants contained between 7-11 mutations away from the WT sequence. The right panel shows the improved binding of a variant with 13 mutations (light grey line) away from the WT sequence (dark grey line). Overall, around 75,000 variants were identified with stronger binding affinity than the initial sequence, while the tightest binding variant exhibited ˜100-fold improved apparent affinity (K_{da}pp) compared to WT as shown in FIG. 5B.

### Example 2: Generation of Polypeptide Fusions, Multiple Epitopic or Specific Polypeptides

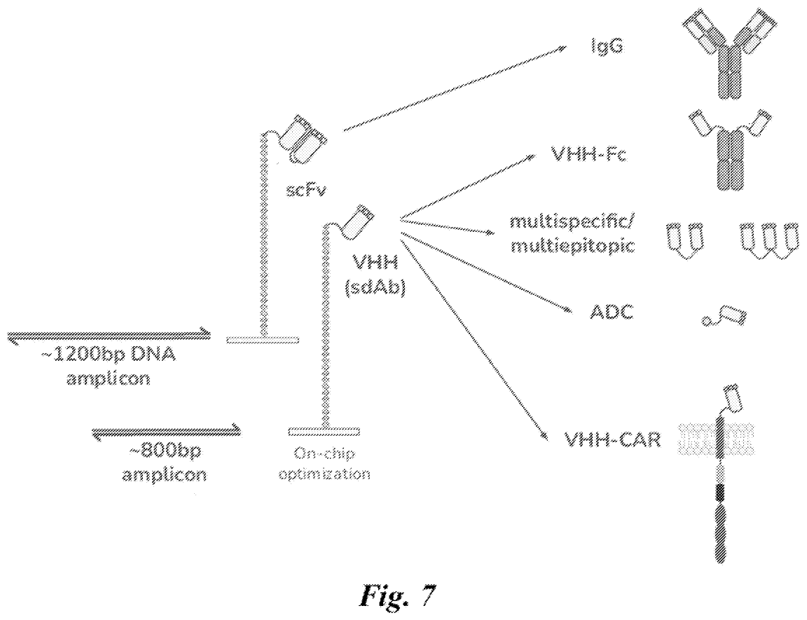

Using similar methods as described in Example 1, more complex polypeptides may be generated based on the quantitative analysis of polypeptide libraries. A first library comprising scFv variants or VHH variants is generated. The first library comprises sub-libraries as described in Example 1, for example, a sub library comprising 20 variant for each residue corresponding to a single amino acid substitution to each canonical amino acid at each residue number. Similarly to Example 1, the library is then subjected to a quantitative binding assay in which labeled antigen of interest is allowed to interact with the polypeptide library. The labeled antigen is added at various concentrations and the intensity of the label is imaged to determine the interaction at each concentration. A binding curve for each polypeptide is generated and fitted to determine a quantitative binding characteristic. Once data relating to the library has been generated, a second library is constructed using the information regarding variants. For example, variants comprising multiple mutation corresponding to combination of variants with neutrals or positive effects can be constructed for the second library. The second library is assayed to identify polypeptide with optimized or improved binding characteristics. These optimized polypeptides may than be used as a core or domain of a new polypeptide construct. Although the library is generated using scFvs or VHHs, larger polypeptides or polypeptide fusions can be generated. FIG. 7 shows a schematic for polypeptide fusion that can be generated. Based on the identification of an optimized scFv, a full IgG antibody can be generated using the sequence information of the optimized scFv and encoding an IgG antibody that comprises the structure or sequence of the optimized scFv. A similar method can be used for a VHH library. As shown in FIG. 7, the sequence of the optimized VHH can be used to construct a VHH-Fc fusion, combined with other VHHs to generate multiple specific or multi epitopic polypeptides, conjugated to a drug to make an antibody-drug conjugate, or combined with a chimeric antigen receptor to make a VHH-CAR. Regarding, multi-specific or multi-epitopic constructs, FIG. 8 shows a Venn diagram of binding to different antigens. The VHHs may be individually assayed for a specific antigen and then combined to allow for multi-specificity.

### Example 3: Generation of Bi-Epitopic Polypeptides

Bi-epitopic polypeptides are a class of antibodies or antibody fragments that are capable of binding two distinct epitopes on the same antigen. A bi-epitopic antibody may have a number of distinct advantages over an antibody that targets a single epitope, including, an increased avidity to the target antigen and a decreased susceptibility to antibody-evading antigen mutations. For example, a bi-epitopic VHH developed by Janssen/Johnson & Johnson obtained FDA approval for use as a BCMA-directed CAR-T cell therapy for the treatment of relapsed/refractory multiple myeloma.

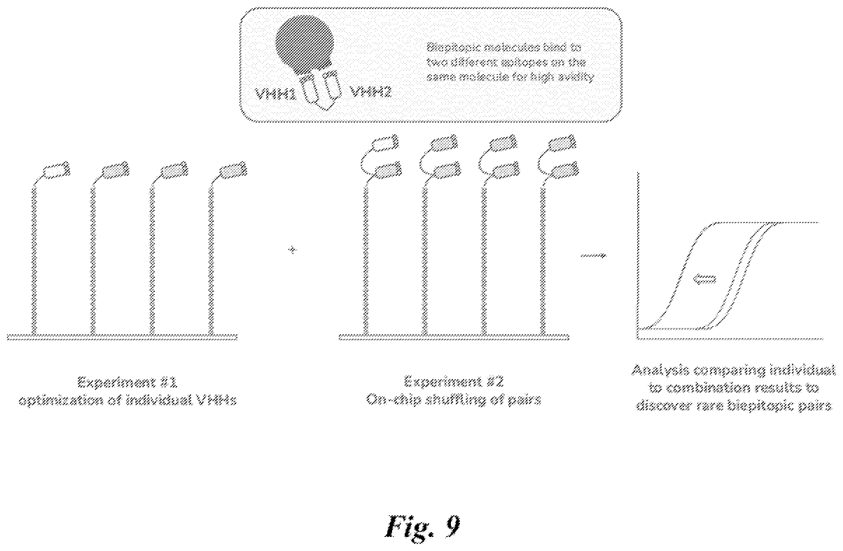

Traditional approaches to develop bi-epitopic antibodies have relied on prior knowledge of antibodies or antibody fragments that bind distinct epitopes on the target antigen or utilized low throughput epitope binning methods to individually screen and discover pairs of antibody fragments that bind distinct epitopes on the same antigen. The Prot-MaP platform enables a systematic, high-throughput approach to screen large libraries of tandemly arrayed VHHs to identify and characterize bi-epitopic tandem VHHs (FIG. 9). The input VHHs into these libraries may be generated in several ways including, but not limited to, DNA synthesis, immunization of animals (alpacas, llamas, rats, mice, among many others) and mining of human immune repertoire sequences.

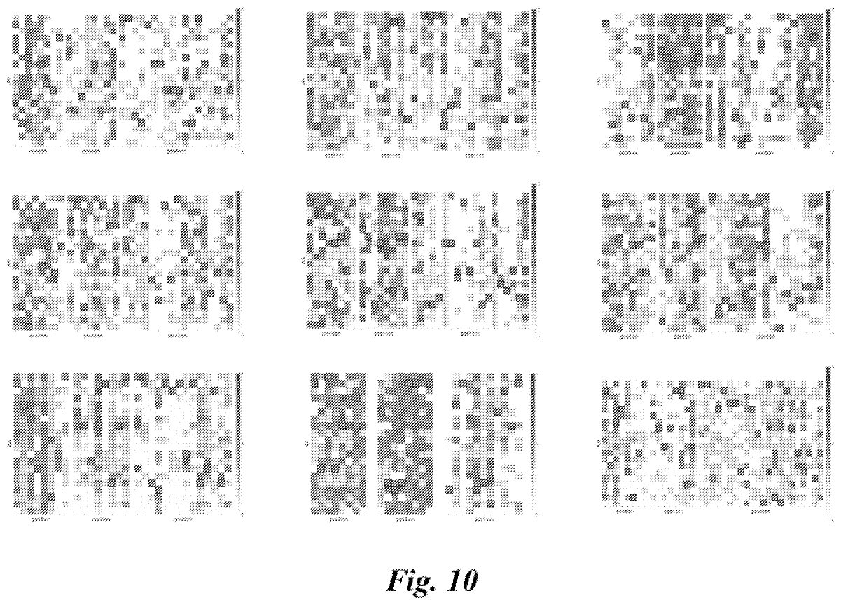

Using publicly available sources, we identified a large set of VHHs targeting SARS-CoV-2 Spike and RBD proteins. In order to verify binding activity of these VHHs to RBD, we first constructed a survey library in which every VHH in the set was placed in the context of a variety of N-terminal linker and C-terminal spacer polypeptides to optimize initial display. From this library, several VHHs (and their associated display contexts) were identified that bound SARS-CoV-2 RBD with moderate to high affinities. Next, in order to optimize affinity of selected VHHs, a library was generated comprising single mutant variants of 14 highest affinity VHHs identified in the previous step, similarly as in Example 1. The library was sequenced and affinities of these variant mutants were quantitatively characterized in a Prot-MaP experiment. A series of fluorescently-labeled SARS-CoV-2 RBD solutions at varying concentrations were sequentially added to the sequencing chip, allowed to bind to the displayed VHH s and imaged. The fluorescent signal from the bound RBD was quantified, fit to binding curves which were used to derive the binding affinities of each displayed VHH to the RBD target, thus generating a single mutant binding affinity landscape that quantitatively described the impact of specific amino acid changes to every residue in the CDRs of each of these VHHs was thus generated. FIG. 10 shows the resultant heat map of binding data for all single mutants from a subset of the 14 VHHs.

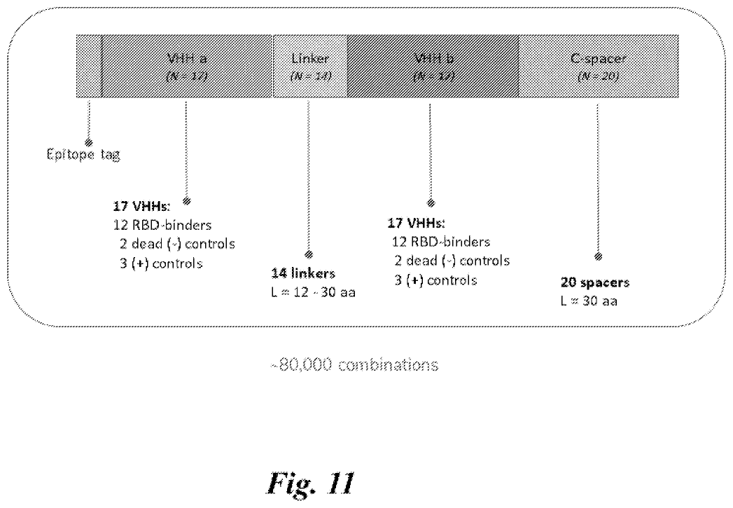

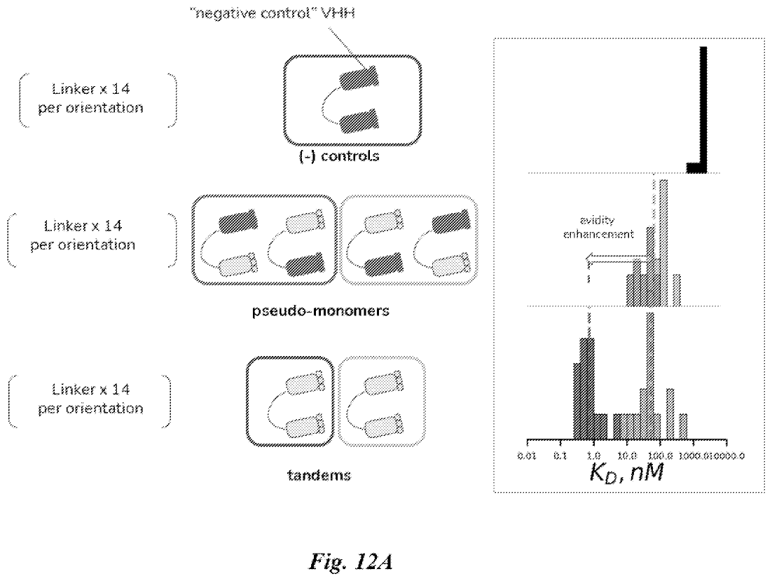

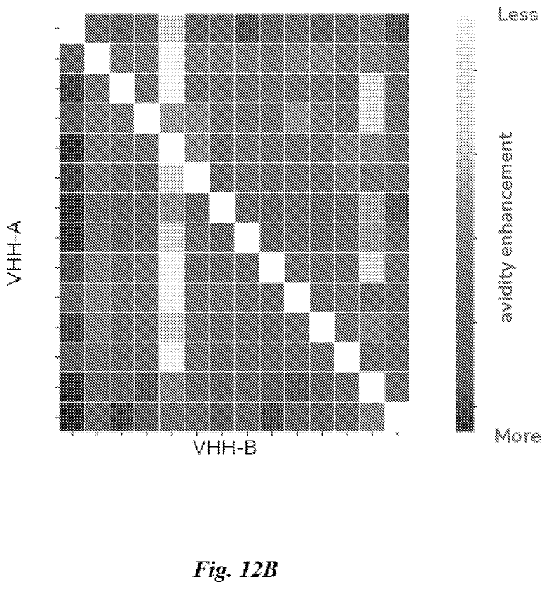

In the next step, the single mutant binding data was used to build two additional libraries. First, in order to interrogate avidity enhancement achieved through tandem presentation of pairs of VHHs, a tandem VHH library was generated. A moderate affinity (Kd ranging from 5-30 nM) single mutant variant was selected from 12 of the 14 VHHs. To this set, 3 positive control VHHs expected to bind SARS-CoV2-RBD and 2 negative control VHHs that were not expected to bind SARS-CoV-2 RBD were added. All possible pairwise combinations of the 17 VHHs with each other connected by a flexible protein linker were then generated. 14 unique linker sequences varying in length (12-30 amino acids), charge, and predicted secondary structure were used to connect each pair of VHHs. Finally, each pair was also embedded in a variety of different C-spacer contexts as described in Example 1 and shown in schematic form in FIG. 11 to yield a library containing >80,000 variants. In order to identify the large avidity increases expected from simultaneous bi-epitopic binding of two high-affinity VHHs, it is necessary to compare the affinities measured for the tandem pairs (the tandem dataset) to the affinities of each component VHH as an individual monomer (the monomer dataset). Though it is more efficient, in principle, to generate both the tandem and monomer datasets together on the same chip (instead of two separate experiments), one of the challenges with doing so is that simultaneously clustering and sequencing libraries of significantly different lengths together often results in large and unpredictable skews in relative representation. To minimize such skews, it is beneficial for library members sequenced together to be of similar lengths, and to this end, we included pseudo-monomer VHHs—comprised of a given VHH and a negative control “dead” VHH arrayed in both orientations (a-b and b-a)—which were used as a proxy for individual, monomer VHHs. The library was sequenced and assayed for binding to SARS-CoV-2 RBD as described above. Tandem VHH pairs in a given orientation that bound the RBD with an affinity significantly larger than the mean affinity of the pseudo-monomer VHHs in the pair, were thus identified (FIG. 12).

Using the single mutant binding data (FIG. 10), a second library was constructed to optimize the affinities of the individual VHHs that formed bi-epitopic tandem pairs. As described in Example 1, an affinity optimization library was generated based on the data from the single mutant library and subjected to a binding assay to identify individual VHHs with improved affinities over the starting variant. (FIG. 13)

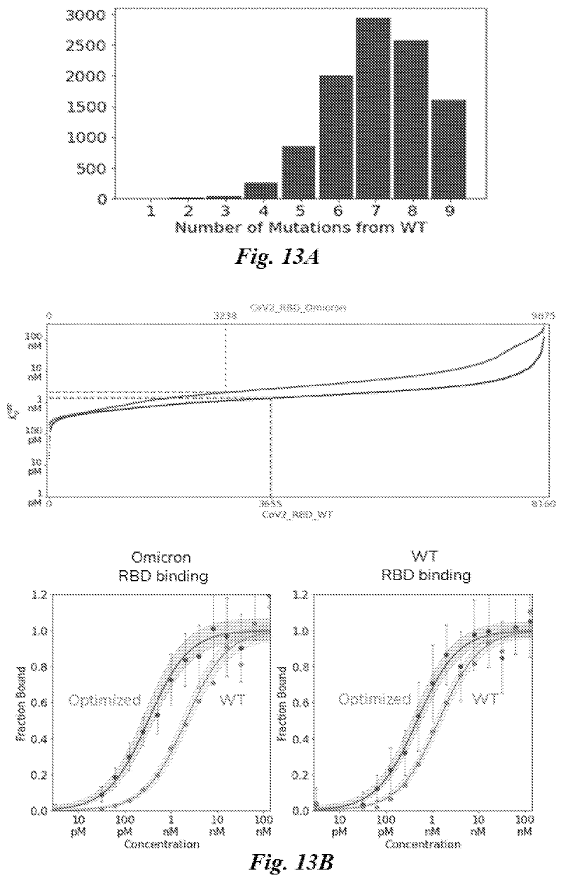

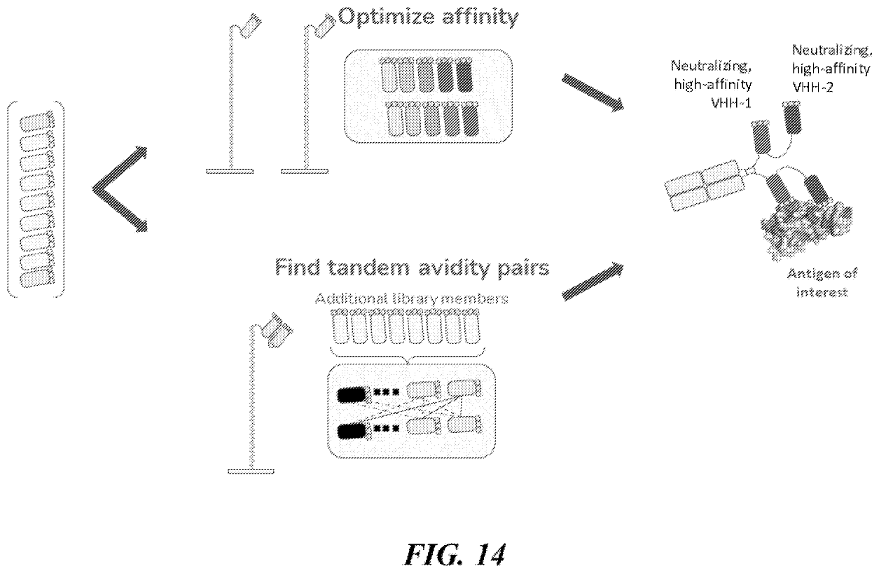

To generate the final affinity- and avidity-enhanced molecules, tandem VHH pairs that showed significant avidity enhancement were reconstructed by replacing the moderate affinity single mutant VHHs in the tandem VHH pair with the optimized tightest binding affinity variant of each VHH (FIG. 14).

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A high throughput method for identifying an optimized polypeptide, comprising:
(a) providing a first library of polynucleotides encoding a first library of variant polypeptides;
(b) processing said first library of polynucleotides to produce said first library of variant polypeptides wherein said variant polypeptides are attached to said first library of polynucleotides;
(c) identifying one or more characteristics comprising an equilibrium binding constant, a kinetic binding constant, a protein stability measurement, an enzyme activity, a fractional activity, a nonspecific binding potential, an aggregation potential, a hydrophobicity, a protein expression level, or a maturation time of at least a portion of said first library of variant polypeptides;
(d) providing a second library of polynucleotides encoding a second library of variant polypeptides selected based at least on one or more characteristic identified in (c);
(e) processing said second library of polynucleotides to produce said second library of variant polypeptides wherein said variant polypeptides are attached to said second library of polynucleotides; and
(f) analyzing said second library of variant polypeptides to produce optimized data.

2. A high throughput method for measuring a characteristic of a polypeptide, comprising:
(a) providing a first library of polynucleotides attached to a solid surface, wherein said library of polynucleotides encode a library of variant polypeptides;
(b) processing said library of polynucleotides to produce said library of variant polypeptides, wherein said variant polypeptides are attached to said library of polynucleotides; and
(c) identifying one or more of characteristics comprising an equilibrium binding constant, a kinetic binding constant, a protein stability measurement, an enzyme activity, a fractional activity, a nonspecific binding potential, an aggregation potential, a hydrophobicity, a protein expression level, or a maturation time of at least a portion of said library of variant polypeptides.

3. A high throughput method for screening a plurality of polypeptides, comprising:
(a) providing a first library of polynucleotides encoding a library of variant polypeptides, wherein said first library of variant polypeptides comprises at least 90% of all single amino acid variants wherein amino acid residues are substituted for an amino acid selected from a set of twenty different amino acids;
(b) processing said first library of polynucleotides to produce said first library of variant polypeptides wherein said variant polypeptides are attached to said first library of polynucleotides; and
(c) identifying one or more characteristics of polypeptides of the first library of variant polypeptides.

4. A high throughput method for screening a plurality of polypeptides, comprising:
(a) providing a first library of polynucleotides encoding a first library of variant polypeptides, wherein said first library of variant polypeptides comprises single amino acid variants polypeptides corresponding to at least 90% of possible single nucleotide variants for a given reference sequence in a reference polypeptide, wherein for a given single amino acid variant, the amino acid residue is substituted for another amino acid selected from a set of twenty different amino acids;
(b) processing said first library of polynucleotides to produce said first library of variant polypeptides wherein said variant polypeptides are attached to said first library of polynucleotides; and
(c) identifying one or more characteristics of polypeptides of the first library of variant polypeptides.

5. The method of claims 3 or 4, wherein said one or more characteristics comprises an equilibrium binding constant, a kinetic binding constant, a protein stability measurement, an enzyme activity, a fractional activity, a nonspecific binding potential, an aggregation potential, a hydrophobicity, a protein expression level, or a maturation time of at least a portion of said first library of variant polypeptides

6. The method of any one of claims 2-5, further comprising: (d) providing a second library of polynucleotides encoding a second library of variant polypeptides selected based at least on one or more characteristic identified in (c); (e) processing said second library of polynucleotides to produce said second library of variant polypeptides wherein said variant polypeptides are attached to said second library of polynucleotides; and (f) analyzing said second library of variant polypeptides to produce optimized data.

7. The method of claim 1 or 6, further comprising (g) identifying an optimized polypeptide based on said optimized data.

8. The method of any one of claims 1-7, wherein said high throughput method does not comprise a cell.

9. The method of any one of claims 1-8, wherein said first library of polynucleotides is a library of deoxyribonucleic acid molecules.

10. The method of any one of claims 1, 2, and 5-9, wherein said equilibrium binding constant is a dissociation constant (K_{d}).

11. The method of any one of claims 1, 2, and 5-9, wherein said equilibrium binding constant is an association constant (Kₐ).

12. The method of any one of claims 1, 2, and 5-11, wherein said kinetic binding constant is an association rate constant (kₒₙ).

13. The method of any one of claims 1, 2, and 5-11, wherein said kinetic binding constant is a dissociation rate constant (k_{off}).

14. The method of any one of claims 1, 2, and 5-13, wherein said protein stability measurement is a protein melting temperature (Tₘ).

15. The method of any one of claims 1, 2, and 5-13, wherein said protein stability measurement is a midpoint denaturtion concentration of a chemical denaturant (Cₘ).

16. The method of any one of claims 1, 2, and 5-15, further comprising in (d), identifying negative variations, positive variations, and neutral variations from said first library of variant polypeptides.

17. The method of claim 16, wherein said neutral variations have a dissociation constant greater than 0.25 times and less than 2 times a dissociation constant of a starting polypeptide.

18. The method of claim 16, wherein said positive variations have a dissociation constant less than or equal to 0.25 times a dissociation constant of a starting polypeptide.

19. The method of claim 16, wherein said negative variations have a dissociation constant greater than or equal to 2 times a dissociation constant of a starting polypeptide.

20. The method of any one of claims 1-19, wherein said first library of variant polypeptides comprises single amino acid variants wherein amino acid residues are substituted for an amino acid selected from a set of amino acids.

21. The method of claim 20, wherein said set of amino acid comprises 10 different amino acids.

22. The method of claim 20, wherein said set of amino acid comprises 20 different amino acids.

23. The method of claim 20, wherein said set of amino acids comprises alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine.

24. The method of any one of claims 1-23, wherein said first library of variant polypeptides consists of variants of a starting polypeptide and said starting polypeptide.

25. The method of claim 24, wherein said first library of variant polypeptides comprises double amino acid variants of interacting amino acid pairs.

26. The method of claim 25, wherein said double amino acid variants of interacting amino acid pairs comprise variants wherein amino acid residues of said interacting amino acid pairs are substituted for all twenty amino acids.

27. The method of claim 26, wherein said interacting amino acid pairs are identified by via a crystal structure of said original polypeptide.

28. The method of claim 27, wherein said interacting amino acid pairs comprise inter-polypeptide interactions and intra-polypeptide interactions.

29. The method of any one of claims 1-28, wherein said first library of variant polypeptides comprises single amino acid insertions at each position.

30. The method of any one of claims 1-29, wherein said first library of variant polypeptides comprises single amino acid deletions.

31. The method of any one of claims 1-30, wherein said first library of variant polypeptides comprises double amino acid deletions.

32. The method of any one of claims 1-31, wherein said first library of variant polypeptides comprises triple amino acid deletions.

33. The method of any one of claims 1-32, wherein said first library of variant polypeptides comprises at least four amino acid deletions.

34. The method of any one of claims 1-33, wherein analyzing said first library of variant polypeptides comprises transcribing and translating a polynucleotide of said first library of variant polynucleotides, wherein said polypeptide encoded by said polynucleotide is attached to said polynucleotide.

35. The method of any one of claims 1, 2, and 5-34, wherein identifying said equilibrium binding constant, kinetic binding constant, protein stability measurement, enzyme activity, fractional activity, nonspecific binding potential, aggregation potential, hydrophobicity, protein expression level, or maturation time comprises performing a binding assay on said first library of variant polypeptides.

36. The method of claim 35, wherein identifying said equilibrium binding constant, kinetic binding constant, protein stability measurement, enzyme activity, fractional activity, nonspecific binding potential, aggregation potential, hydrophobicity, protein expression level, or maturation time comprises sequencing said first library of polynucleotides and associating sequences of said first library of polynucleotides with said binding assay.

37. The method of claim 35, wherein said binding assay comprises assaying binding of said first library of variant polypeptides to an antigen.

38. The method of claim 35, wherein said binding assay comprises assaying binding of said first library of variant polypeptides to more than one antigen.

39. The method of claim 38, wherein said binding assay comprises assaying binding of said first library of variant polypeptides to a plurality of antigens.

40. The method of claim 39, further comprising identifying a variant polypeptide that binds to two or more antigens of said plurality of antigens.

41. The method of claim 39, further comprising identifying a variant polypeptide that binds to at least one antigen of said plurality of antigens and does not bind to a different antigen of said plurality of antigens.

42. The method of claim 39, further comprising identifying a variant polypeptide that does not bind to said plurality of antigens.

43. The method of claim 1, 2, and 5-38, wherein said identifying said equilibrium binding constant, kinetic binding constant, protein stability measurement, enzyme activity, fractional activity, nonspecific binding potential, aggregation potential, hydrophobicity, protein expression level, or maturation time comprises generating binding data for more than one target.

44. The method of claim 43, wherein said second library is generated based at least on binding data for more than one target.

45. The method of any one of claims 1 and 6-44, wherein processing said second library of variant polypeptides comprises transcribing and translating a polynucleotide of said second library of variant polynucleotides, wherein said polypeptide encoded by said polynucleotide is attached to said polynucleotide.

46. The method of any one of claims 1 and 6-45, wherein said identifying said optimized polypeptide comprises performing a binding assay on said second library of variant polypeptides encoded by said second library of polynucleotides.

47. The method of claim 46, wherein identifying said equilibrium binding constant, kinetic binding constant, protein stability measurement, enzyme activity, fractional activity, nonspecific binding potential, aggregation potential, hydrophobicity, protein expression level, or maturation time comprises sequencing said second library of polynucleotides and associating sequences of said second library of polynucleotides with said binding assay.

48. The method of any one of claims 1 and 6-47, wherein said second library of variant polypeptides comprises at least 10⁴ polypeptides.

49. The method of any one of claims 1-48, wherein said first library of polynucleotides comprises at least 10⁶ polynucleotides.

50. The method of any one of claims 1-49, wherein said first library of variant polypeptides comprises at least 10⁴ polypeptides.

51. The method of any one of claims 1-50, wherein said method is performed in less than 48 hours.

52. The method of any one of claims 1-51, wherein said first library of variant polypeptides comprises a library of individual VHH antibodies.

53. The method of claim 52, wherein said second library of variant polypeptides comprises a library of VHH antibody fusions.

54. The method of any one of claims 1-53, wherein said first library of variant polypeptides comprises a library of individual single chain variable fragments (scFvs).

55. The method of claim 54, wherein said second library of variant polypeptides comprises a library of individual single chain variable fragments (scFvs) fusions.

56. A high throughput method for identifying an optimized polypeptide, comprising:
(a) obtaining a dataset comprising binding data of an antigen to a first plurality of polypeptides and providing a plurality of polynucleotides based at least in part on said dataset;
(b) providing a plurality of polynucleotides attached to a solid surface;
(c) processing said plurality of polynucleotides to produce a second plurality of polypeptides;
(d) exposing an antigen to said second plurality of polypeptides and detecting an interaction of at least one polypeptide of said second plurality of polypeptides with said antigen;
(e) generating sequence data comprising (i) a sequence of at least the at least one polypeptide, or (ii) a sequence of the corresponding polynucleotide that encodes said at least one polypeptide;
(f) based at least in part on sequence data and said detecting, generating a plurality of fusions polypeptides wherein a fusion polypeptide of said plurality of fusion polypeptides comprises a polypeptide from each of said first plurality of polypeptides or said second plurality of polypeptides capable of binding said antigen; and
(g) repeating (a) through (e), wherein said dataset comprises binding data of an antigen to said plurality of polypeptide fusions to identify said optimized polypeptide.

57. A method for identifying an optimized polypeptide, comprising:
(a) providing a plurality of polynucleotides attached to a solid surface wherein said plurality of polynucleotides encode a plurality of fusion polypeptides, wherein a fusion polypeptide of said plurality of fusion polypeptides comprises two or more domains;
(b) processing said plurality of polynucleotides to produce a plurality of fusion polypeptides;
(c) exposing an antigen to said plurality of fusion polypeptides and detecting an interaction of at least one fusion polypeptide of said plurality of fusion polypeptides with said antigen;
(d) generating sequence data comprising (i) a sequence of at least the at least one fusion polypeptide, or (ii) a sequence of the corresponding polynucleotide that encodes said at least one fusion polypeptide; and
(e) based at least in part on said sequence data, said detecting, and a dataset comprising binding data of an antigen to a plurality of single domain polypeptides, generating an optimized polypeptide capable of binding said antigen.

58. The method of claim 56, wherein said dataset is generated by identifying a polypeptide of said first plurality of polypeptides that can interact with said antigen.

59. The method of claim 56 or 58, wherein said dataset is generated at least by exposing said antigen to said first plurality of polypeptides and detecting an interaction of at least one polypeptide of said first plurality of polypeptides with said antigen.

60. The method of claim 59, wherein the first plurality of polypeptides is generated by (i) providing a plurality of first polynucleotides encoding a plurality of first polypeptides; (ii) providing a plurality of first capture probes attached to a solid surface configured to anneal to said first plurality of polynucleotides to produce a plurality of captured polynucleotides; (iii) processing said plurality of captured polynucleotides to produce said first plurality of polypeptides.

61. The method of any one of claims 56 and 58-60, wherein the data pertaining to first plurality of polypeptides comprises sequence data generated at least by sequencing said plurality of captured polynucleotides, wherein said plurality of capture polynucleotides is a plurality of VHH polynucleotides.

62. The method of any one of claims 56-61, wherein detecting said interaction of at least one polypeptide of said plurality of polypeptides with said antigen comprises identifying a quantitative characteristic of said polypeptide.

63. The method of claim 62, wherein identifying said quantitative characteristic of said polypeptide further comprises identifying said polypeptide as comprising one or more of a negative, neutral or positive mutation.

64. The method of any one of claims 56 and 58-63, wherein said plurality of fusion polypeptides comprises a polypeptide for at least 50%, 60%, 70%, 80%, 90%, or more, of all possible fusion pair combinations or permutations of said polypeptides of said first plurality of polypeptides.

65. The method of any one of claims 56 and 58-63, wherein said plurality of fusion polypeptides comprises a polypeptide for of all possible fusion pair combinations or permutations of said polypeptides of said first plurality of polypeptides.

66. The method of claim 57, wherein said dataset comprises data corresponding to single domain polypeptides that correspond to one or domains of the fusion polypeptides.

67. The method of claim 57 or 66, wherein said dataset is generated by identifying a single domain polypeptide that can interact with said antigen.

68. The method of any one of claims 57 and 66-67, wherein dataset is generated at least by exposing said antigen to a plurality of single domain polypeptides and detecting an interaction of at least one single domain polypeptide of said plurality of single domain polypeptides with said antigen.

69. The method of any one of claims 57 and 66-68, wherein the plurality of single domain polypeptides is generated by (i) providing a plurality of single domain polynucleotides encoding a plurality of single domain polypeptides, wherein said single domain polynucleotides are coupled to a solid surface; (iii) processing said plurality of single domain polynucleotides to produce said plurality of single domain polynucleotides polypeptides.

70. The method of any one of claims 57 and 66-69, wherein the dataset comprises sequence data generated at least by sequencing said plurality of single domain polynucleotides.

71. The method of any one of claims 57 and 66-70, wherein said single domain polypeptide comprises a VHH.

72. The method of any one of claims 57 and 66-71, wherein said fusion polypeptide comprises a VHH-VHH fusion.

73. The method of any one of claims 57 and 66-72, wherein said plurality of fusion polypeptide comprise a sequence corresponding to one or more polypeptide of said plurality of single domain polypeptides.

74. The method of any one of claims 57 and 66-73, wherein a fusion polypeptide of said plurality of fusion peptides comprises sequences of two polypeptides of said plurality of single domain polypeptides.

75. The method of any one of claims 57 and 66-74, wherein said plurality of fusion polypeptides comprises a polypeptide for at least 50%, 60%, 70%, 80%, 90%, or more, of all possible fusion pair combinations or permutations of said single domain polypeptides of said plurality of single domain polypeptides.

76. The method of any one of claims 57 and 66-75, wherein said plurality of fusion polypeptides comprises a polypeptide for of all possible fusion pair combinations or permutations of said single domain polypeptides of said plurality of single domain polypeptides.

77. The method of any one of claims 57 and 66-76, wherein said plurality of single domain polypeptides comprises a plurality of single domain polypeptides differing by a single point mutation.

78. The method of any one of claims 57 and 66-77, wherein said plurality of single domain polypeptides comprises a plurality of single domain polypeptides differing by a single point mutation in a binding interface.

79. The method of any one of claims 57 and 66-77, wherein said plurality of single domain polypeptides comprises a plurality of single domain antibody fragments differing by a single point mutation in a CDR.

80. The method of any one of claims 57 and 66-79, wherein said plurality of single domain polypeptides comprises a plurality of 20 polypeptides wherein a different amino acid is encoded at a given residue.

81. The method of any one of claims 57 and 66-80, wherein detecting said interaction of at least one single domain polypeptide of said plurality of single domain polypeptides with said antigen comprises identifying a quantitative characteristic of said single domain polypeptide.

82. The method of any one of claims 57 and 66-81, wherein identifying said quantitative characteristic of said polypeptide further comprises identifying said single domain polypeptide as comprising one or more of a negative, neutral or positive mutation.

83. The method of any one of claims 57 and 66-82, wherein detecting said interaction of at least one fusion polypeptide of said plurality of fusion polypeptides with said antigen comprises identifying a quantitative characteristic of said fusion polypeptide.

84. The method of any one of claims 57 and 66-83, wherein identifying said quantitative characteristic of said polypeptide further comprises identifying said fusion polypeptide as comprising a bi-epitopic interaction.

85. The method of claim 84, wherein identifying said fusion polypeptide as comprising an avidity-enhanced interaction comprises comparing said quantitative characteristic of said fusion polypeptide with quantitative characteristics of a first single domain or a second single domain, wherein the sequence of said fusion polypeptide comprises the sequence of said first single domain and said second single domain.

86. The method of claim 85, wherein said avidity-enhanced interaction is identified when said quantitative characteristic of said fusion polypeptide is greater than said quantitative characteristics of said first single domain or said second single domain.

87. The method of claim 85, wherein said optimized polypeptide comprises additional mutations of said fusion polypeptide identified as comprising an avidity-enhanced interaction, wherein said mutation increases the binding affinity of said fusion polypeptide to said antigen.

88. The method of any one of claims 57 and 66-87, wherein said data comprising binding data of an antigen to a plurality of the single domain polypeptides is obtained at a same time as (c) or (d) is performed.

89. The method of any one of claims 57 and 66-88, wherein said data comprising binding data of an antigen to a plurality of the single domain polypeptides is obtained prior to (a), and wherein said providing said plurality of polynucleotides attached to a solid support is based at least in part on said dataset

90. The method of any one of claims 57 and 66-89, wherein said plurality of fusion polypeptides comprise sequences of single domain polypeptides comprising a moderate affinity to said antigen.

91. The method of any one of claims 57 and 66-90, wherein said plurality of fusion polypeptides comprise sequences of single domain polypeptides comprising minimal affinity or no affinity to said antigen.

92. The method of claim 91, wherein said sequences of single domain polypeptides comprising minimal affinity or no affinity comprise a substantially similar size or length to a single domain polypeptide that is capable of binding said antigen.

93. The method of claim 91, wherein said sequences of single domain polypeptides comprising minimal affinity or no affinity comprise no more than a 10% difference in size or length to a single domain polypeptide that is capable of binding said antigen.

94. The method of any one of claims 57 and 66-91, wherein a single domain polypeptide of said plurality of single domain polypeptides comprises a N-terminal linker or a C-terminal spacer.

95. The method of any one of claims 57 and 66-94, wherein a single domain polypeptide of said plurality of single domain polypeptides comprises a N-terminal linker and a C-terminal spacer.

96. The method of any one of claims 57 and 66-95, wherein said plurality of single domain polypeptides comprises a plurality of different N-terminal linker sequences and different C-terminal spacer sequences.

97. The method of any one of claims 56-96, wherein said dataset is derived from data in a public database.

98. The method of any one of claims 56-97, wherein said fusion polypeptide is a polypeptide-Fc fusion.

99. The method of claim 98, wherein said polypeptide-Fc fusion comprises an antibody fragment crystallization region (Fc region) capable of binding said antigen.

100. The method of any one of claims 56-99, wherein said fusion polypeptide comprises a chimeric antigen receptor.

101. The method of any one of claims 56-100, wherein said fusion polypeptide comprises a VHH nanobody.

102. The method of any one of claims 56-101, wherein said fusion polypeptide comprises a pair of bivalent VHH nanobodies.

103. The method of any one of claims 56-101, wherein said fusion polypeptide comprises a pair of bi-epitopic VHH nanobodies.

104. The method of any one of claims 56-101, wherein said fusion polypeptide comprises multivalent VHH nanobodies.

105. The method of any one of claims 56-104, wherein said fusion polypeptide comprises a linker connecting a first domain of the fusion polypeptide and a second domain of the fusion polypeptide.

106. The method of claim 105, wherein said first domain comprises a VHH.

107. The method of claim 105 or 106, wherein said second domain comprises a VHH.

108. The methods of any one of claims 105-107 wherein said first domain comprises a first VHH and said second domain comprise a second VHH.

109. The method of any one of claims 105-108, wherein said first VHH and said second VHH bind a same antigen.

110. The method of claim 109, wherein said same antigen comprises a polypeptide, lipid, or carbohydrate, or cell.

111. The method of any one of claims 105-110, wherein said linker comprises at least 12 amino acids.

112. The method of any one of claims 105-110, wherein said linker comprises at least 20 amino acids.

113. The method of any one of claims 105-110, wherein said linker comprises at least 30 amino acids.

114. The method of any one of claims 105-113, wherein said linker comprises a net positive charge.

115. The method of any one of claims 105-113, wherein said linker comprises a net negative charge.

116. The method of any one of claims 105-113, wherein said linker comprises a net neutral charge.

117. The method of any one of claims 56-116, wherein said plurality of polynucleotides comprises at least 10⁴ polynucleotides.

118. The method of any one of claims 56-117, wherein said optimized polypeptide comprise an increased avidity effect.

119. The method of any one of claims 56-118, wherein prior to (a) said solid surface comprises plurality of capture oligonucleotides configured to anneal to a plurality of precursor polynucleotides, and wherein said plurality of precursor polynucleotides anneal to said plurality of capture nucleotide thereby producing said plurality of polynucleotides attached to a solid surface.

120. The method of claim 119, wherein said producing said plurality of polynucleotides attached to a solid surface comprises an amplification or extension of said plurality of precursor polynucleotides.

121. The method of claim 120, wherein said amplification comprises bridge amplification.

122. The method of any one of claims 56-121, wherein said solid support comprises a bead.

123. The method of any one of claims 56-122, wherein said solid support comprises sequencing flow cell.

124. The method of any one of claims 56-123, wherein (d) comprises sequencing said plurality of polynucleotides.

125. The method of claim 124, wherein (e) comprises generating said optimized polypeptide based at least in part on said sequence data generated from of said sequencing of said plurality of polynucleotides and said detecting.

126. The method of any one of claims 56-125, wherein a fusion polypeptide of said plurality of fusion polypeptides comprises a N-terminal linker or a C-terminal spacer.

127. The method of any one of claims 56-126, wherein a fusion polypeptide of said plurality of fusion polypeptides comprises a N-terminal linker and a C-terminal spacer.

128. The method of any one of claims 56-127, wherein a fusion polypeptide comprises a plurality of different N-terminal linker sequences and different C-terminal spacer sequences.

129. The method of any one of claims 56-128, wherein said optimized polypeptide comprises a bi-epitopic polypeptide.

130. The method of any one of claims 56-128, wherein said optimized polypeptide comprises a tri-epitopic polypeptide.

131. The method of any one of claims 56-128, wherein said optimized polypeptide comprises a tetra-epitopic polypeptide.

132. The method of any one of claims 56-128, wherein said optimized polypeptide comprises a multimeric polypeptide.

133. The method of any one of claims 56-132, wherein said optimized polypeptide comprises at two or more domains capable of binding to said antigen, wherein at least two domains are identical.

134. The method of any one of claims 56-133, wherein said optimized polypeptide comprises two or more domains capable of binding to said antigen, wherein the two or more domains are different from one another.

135. A method for identifying a bi-epitopic polypeptide, comprising:
(a) providing a plurality of polynucleotides attached to a solid surface, wherein said plurality of polynucleotides encoding a plurality of VHH polypeptides;
(b) processing said plurality of polynucleotides to produce said plurality of VHH polypeptides;
(c) exposing an antigen to said plurality of polypeptides and detecting an interaction of at least one VHH polypeptide of said plurality of VHH polypeptides with said antigen;
(d) sequencing said plurality of polynucleotides;
(e) providing a second plurality of polynucleotides attached to a solid surface, wherein said second plurality of polynucleotides encode a plurality of VHH-VHH fusion polypeptides;
(f) processing said plurality of second polynucleotides to produce a plurality of VHH-VHH fusion polypeptides;
(g) exposing an antigen to said plurality of VHH-VHH fusion polypeptides and detecting an interaction of at least one VHH-VHH fusion polypeptide of said plurality of VHH-VHH fusion polypeptides with said antigen;
(h) sequencing said second plurality of polynucleotides; and
(i) based at least in part on sequence data generated from of said sequencing of (d) and (e) and said detecting of (c) and (g), generating a bi-epitopic polypeptide capable of binding said antigen.

136. A method for generating an optimized polypeptide comprising:
(a) providing a plurality of polypeptides displayed on a solid substrate, wherein a polypeptide of said plurality of polypeptides comprises a binding domain, and one or more of a (i) N-terminal spacer, (ii) a C-terminal spacer, wherein the plurality of polypeptides comprises polypeptides comprising different combinations of N-terminal spacer sequences and C-terminal spacer sequences;
(b) observing a signal of least two polypeptides of said plurality of polypeptides, wherein the signal corresponds to (i) a binding interaction of a polypeptide and an antigen or (ii) a physical characteristic of a polypeptide;
(c) comparing the signals of said at least two polypeptide and determining the combination of N-terminal spacer sequences and C-terminal spacer sequences that generates a target signal.

137. The method of claim 136, wherein said N-terminal spacer or C-terminal spacer does not bind to said antigen.

138. The method of claim 136 or 137, wherein said target signal comprises a signal below a threshold level.

139. The method of any one of claims 136-138, wherein said target signal comprises a signal above a threshold level.

140. The method of any one of claims 136-139, wherein said target signal comprises a highest signal of signals of the plurality of polypeptides.

141. The method of any one of claims 136-140, wherein said target signal comprises a lowest signal of signals of the plurality of polypeptides.

142. The method of any one of claims 136-141, wherein said signal correspond to an equilibrium binding constant, kinetic binding constant, protein stability measurement, enzyme activity, fractional activity, nonspecific binding potential, aggregation potential, hydrophobicity, protein expression level, or maturation time of a polypeptide.

143. A method for discovery of improved pairs of binders comprising:
(a) providing a comprehensive dataset comprising (i) measured quantitative binding characteristics for a plurality of polypeptides comprising two domains, wherein the two domains are independently selected from a set of monomeric domains, wherein the plurality of polypeptides comprise all possible pairs of monomeric polypeptides; and (ii) measured quantitative binding characteristics of each monomeric domain of said set of monomeric domains as an individual monomer polypeptide
(b) comparing values of (i) and (ii) to identify polypeptides comprising improved pairs of binders that exhibit quantitative binding characteristics significantly greater than the binding characteristics of either component individual monomer polypeptide.

144. The method of claim 143, wherein the improved pairs of binders are bi-epitopic binders.

145. The method of claim 143 or 144, wherein said comprehensive data set comprises measured quantitative binding characteristics for set of individuals monomer polypeptides and measured quantitative binding characteristics for at least 50%, 60%, 70%, 80%, 90%, or more, of all possible tandem pair combinations of said set of individual monomer polypeptides.

146. The method of any one of claims 143-145, wherein said comprehensive data set comprises measured quantitative binding characteristics for set of individuals monomer polypeptides and measured quantitative binding characteristics for all possible tandem pair combinations of said set of individual monomer polypeptides.

147. A high throughput method for identifying affinity—and avidity—optimized tandem polypeptides, comprising:
(a) providing a first library of polynucleotides encoding a first library of monomeric variant polypeptides;
(b) processing said first library of polynucleotides to produce said first library of variant polypeptides wherein said variant polypeptides are attached to said first library of polynucleotides;
(c) analyzing said first library of variant polypeptides to produce data;
(d) identifying the binding affinity of at least a portion of said first library of variant polypeptides based on said data;
(e) providing a second library of second polynucleotides encoding a second library of monomeric variant polypeptides from the first library based on the binding data from the first library;
(f) providing a third library of polynucleotides encoding a plurality of tandem polypeptides comprising different combinations of the monomeric variant polypeptides corresponding to the first library, wherein a tandem polypeptide of the plurality of tandem polypeptide comprises a first monomeric variant polypeptide and a second monomeric variant polypeptide.
(g) processing said second and third libraries of polynucleotides to produce said second and third libraries of variant polypeptides wherein said variant polypeptides are attached to said second and third library of polynucleotides;
(h) analyzing said second and third libraries of variant polypeptides to identify affinity enhancing monomer polypeptide variants and avidity-enhancing tandem polypeptides; and
(i) combining avidity and affinity enhancements identified in said second and third libraries by substituting the individually optimized monomers identified in the second library into the corresponding positions in the avidity-enhancing tandem pairs discovered from the second library.

148. The method of claim 147, where the third library comprises a plurality of polypeptides comprising a different linker between the first monomeric variant polypeptide and the second monomeric variant polypeptide.

149. The method of claim 147 or 148, where third library comprises monomeric variants polypeptides comprising a reduced affinity compared to a reference polypeptide based on the binding data from the first library.

150. A composition comprising: an array of polypeptides displayed on a solid surfaces, wherein each polypeptide is co-localized to a corresponding polynucleotide that encode the polypeptide, wherein a polypeptide of said plurality of polypeptides comprises a first domain and a second domain, wherein said first domain and second domain are linked via a linker, wherein the first domain binds a first epitope and the second domain binds a second epitope, wherein the first epitope and second epitope are different.
